(19) United States
(12) Patent Application Publication
(10) Pub. No.: US 2010/0255458 A1
(43) Pub. Date: Oct. 7, 2010

(54) BIOREACTOR

(76) Inventor: Christopher Piper Toby Kinkaid, Portland, OR (US)

Correspondence Address: WILSON, SONSINI, GOODRICH & ROSATI, 650 PAGE MILL ROAD, PALO ALTO, CA, 94304-1050, US

(21) Appl. No.: 12/665,032

(22) PCT Filed: Jun. 18, 2008
(86) PCT No.: PCT/US08/67273

§ 371 Date: Jun. 7, 2010

Related U.S. Application Data
(60) Provisional application No. 60/936,642, filed on Jun. 20, 2007.
(60) Provisional application No. 61/003,575, filed on Nov. 19, 2007.
(60) Provisional application No. 61/052,562, filed on May 12, 2008.

(51) Int. Cl. C12Q 3/00; C12M 1/42; C12N 1/12; C12N 1/20; C12N 1/10

(52) U.S. Cl. 435/3; 435/292.1; 435/257.1; 435/252.1; 435/258.1

(57) ABSTRACT

The invention provides devices and methods for the growth of photoautotrophic organisms. The devices and methods address issues related to the design of bioreactors, selection of a photoautotrophic organism, growth of the photoautotrophic organisms, extraction of biomass products, and/or use of the biomass products as a renewable energy source.

### CROSS-REFERENCE TO RELATED APPLICATION

This application claims the benefit of U.S. Provisional Patent Application No. 60/936,645, filed Jun. 22, 2007, and U.S. Provisional Patent Application No. 61/003,575, filed Nov. 19, 2007, and U.S. Provisional Patent Application No. 61/052,562, filed May 12, 2008, which are incorporated herein by reference in their entirety.

### BACKGROUND OF THE INVENTION

The search for alternative and renewal sources of energy has become increasingly important in recent years. The price of crude oil has increased more than five times in the last six years, resulting in a significant economic strain to many regions. The increase in crude oil price is expected to continue as the oil reserves are diminishing and the demand for energy is expanding throughout the world.

As the cost of fossil energy increases, producing renewable energy using alternative means is becoming more commercially attractive. Renewable energy can include any energy produced from resources like biomass, solar energy, geothermal energy, hydropower, or wind. Of these types of alternative energy sources, production of biomass using photoautotrophic organisms provides an additional benefit in that the biomass can be utilized for producing other high value products. For example, algae can be grown for the production of oil, DHA, and press cake. While the oil and press cake can be utilized for energy generation by combustion, DHA is a high value product that can be utilized for nutraceutical formulations.

However, the economic viability of growing photoautotrophic organisms for production of energy and other high value products is still hampered due to limitations in at least three different areas. These areas include growth of the photoautotrophic organism, extraction of biomass products, and use of the biomass products as a renewable energy source.

### SUMMARY OF THE INVENTION

There is a considerable need for methods and devices that utilize photoautotrophic organisms for a robust production of biomass and/or industrial chemicals that can be used as renewable energy products. The methods and devices of the present invention can be utilized in growth of photoautotrophic organisms, harvest of photoautotrophic organisms, and/or extraction of biomass products from photoautotrophic organisms.

One embodiment of the invention provides for a bioreactor comprising a container for culturing a photoautotrophic organism, said photoautotrophic organism having at least one light absorption pigment, the at least one light absorption pigment having one or more peak absorption wavelengths; and a light source configured to emit one or more wavelengths of light reaching said container, wherein the one or more wavelengths of light are adjustable based on a growth profile of said photoautotrophic organism. Where desired, the light source is configured to emit pulses of light or is placed on the exterior of the reactor. The pulses of light can be adjusted based on the growth profile of said photoautotrophic organism. Optionally, the one or more wavelengths comprise wavelengths between 300 nm and 800 nm.

Where desired, temperature or pH of the bioreactor is controlled based on the growth profile of said photoautotrophic organism. The growth profile can be represented in dry cell weight of said phototrophic organism per volume or in optical density of said phototrophic organism in said bioreactor over a period of time.

In some embodiments of the invention, a bioreactor comprises a container comprising a light-receiving element configured to receive solar light for culturing a photoautotrophic organism and a light conducting channel operably linked to said light-receiving element, wherein said light conducting channel having a surface area that distributes light from at least about 50% of exterior surface area of said channel. The light conducting channel can comprise a glass, a plastic, a polymer, or a reflective element. The light conducting channel can be substantially rod-like or box-like in shape. Optionally, the light conducting channel is placed on the interior of said container. Where desired, the reflective element is positioned at the end of the light conducting channel.

In other embodiments of the invention, a bioreactor comprises a container for culturing a photoautotrophic organism, wherein said container comprises a movable unit mounted therein, wherein said movable unit is adapted to translate horizontally or vertically along a length of said container, wherein said translation concentrates said photoautotrophic organism on one side of said movable unit; and a harvest port extending from said length of said container to collect said concentrated photoautotrophic organism. The movable unit can comprise a perforated material or a mesh. The concentrated photoautotrophic organism can be suspended in a liquid. The bioreactor can further comprise a light conducting channel or a cleaning element mounted on the movable unit. Optionally, the bioreactor can further comprise a light source configured to emit one or more wavelengths of light reaching said container, wherein the one or more wavelengths of light are adjustable based on a growth profile of said photoautotrophic organism.

In one aspect of the invention, a bioreactor comprises a container for culturing a photoautotrophic organism, a light source configured to emit one or more wavelengths of light that reaches said container to support growth of said photoautotrophic organism, and an energy converter for production of electrical energy from a renewable energy source, wherein said energy converter is operably linked to said light source. The energy converter can be selected from the group consisting of a solar panel, a wind turbine, a combustion device, a steam turbine, a dam, and any combination thereof. Optionally, the renewable energy source is solar energy.

In some embodiments of the invention, the renewable energy source is selected from the group consisting of wind energy, hydroelectric energy, biomass energy, and thermal energy. The production of electrical energy can be carbon neutral. The bioreactor can further comprise an energy conditioning device or an energy storing device.
The light source can be configured to emit pulses of light. The pulses of light can be adjusted based on the growth profile of said photoautotrophic organism. The container can be made of a material selected from the group consisting of metal, glass, semi-conductor, polymer, and any combination thereof.

In some embodiments of the invention, a bioreactor comprises a container comprising a light-receiving element configured to receive solar light for culturing a photoautotrophic organism during day time and a light source configured to emit one or more wavelengths of light reaching said container to maintain growth of said photoautotrophic organism in the absence of said solar light. The light-receiving element can comprise one or more of the following: an optical fiber cable, a light collecting dish, a window, a parabolic trough concentrator, or a non-imaging optical device. The non-imaging optical device can be a compound parabolic concentrators (CPC).

The concentration ratio of the non-imaging optical device can be between approximately 0.5 to 6, approximately 1.3 to 5, or approximately 2.

In other aspects of the invention, a bioreactor comprises a container for culturing a photoautotrophic organism, said container comprising a reflective element to substantially preclude light loss from or due to said container, and a light source configured to emit one or more wavelengths of light reaching said container to support growth of said photoautotrophic organism. The reflective element can be immobilized to the interior or exterior of said container.

The present invention provides for an array of light sources comprising one or more light sources configured to emit at least one or more wavelengths of light, wherein light emission from said array of light sources is configured to be adjustable to match one or more peak absorption wavelengths of a light absorption pigment that is contained in a photoautotrophic organism. The one or more light sources can be configured to be adjustable to emit all peak absorption wavelengths of said light absorption pigment. Optionally, the one or more light sources is configured to emit one or more wavelengths of light between 300 nm and 800 nm. The array of light sources can comprise a light conducting channel for transmitting photons emitted from said light sources.

Where desired, the light sources can be mounted inside the light conducting channel. Alternatively, the light sources can be mounted outside the light conducting channel. The light sources can be configured to emit pulses of light.

The present invention provides for a manufacturing plant for production of biomass and electric energy comprising a bioreactor for production of biomass, said biomass comprising a photoautotrophic organism and a container, and a power plant operably linked to said bioreactor, wherein said power plant converts said biomass to electricity and carbon dioxide, wherein said carbon dioxide is supplied to said bioreactor for production of said biomass. The bioreactor can comprise a light source configured to emit one or more wavelengths reaching said container, wherein the one or more wavelengths of light are adjustable based on a growth profile of said photoautotrophic organism.

In any of the bioreactors described herein, the bioreactor may also comprise components selected from the group consisting of a baffle, a mixer; a gas supply, a gas sparger, a pressure relief valve, a condenser, a heater, a cooling jacket, a viewing window, and an optical light distributor. The gas sparger can comprise one or more with holes less than approximately 0.01, 0.05, 0.1, 0.25, 0.5, or 1 cm in diameter configured to deliver a gas to the bioreactor. The bioreactors can comprise a container of a material selected from the group consisting of metal, glass, semi-conductor, polymer, and any combination thereof. The photoautotrophic organism grown in the bioreactor can be selected from the group consisting of algae, bacteria, euglena, diatom, and phytoplankton. Where desired, the algae is botryococcus braunii, chlorella, or dunaliella. The light source can be selected from the group consisting of a light emitting diode, a laser, an incandescent light bulb, a gas discharge bulb. Optionally, the light source can be a solar light source. The one or more wavelengths emitted by a light source may correspond to the one or more peak absorption wavelengths of the at least one light absorption pigment of said photoautotrophic organism.

Other aspects of the invention provide methods of growing photoautotrophic organisms, harvesting the photoautotrophic organisms, and/or extracting biomass products from the photoautotrophic organisms.

One embodiment of the invention provides for a method of producing biomass comprising culturing a photoautotrophic organism in a medium contained in a bioreactor operably linked to a light source that emits photons to support growth of said photoautotrophic organism, wherein the light source is configured to yield a biomass production efficiency at no less than about 50, 5, 0.5, 0.05, or 0.005 milligrams of said biomass per kJ of energy that is supplied to the light source. Optionally, the photoautotrophic organism is genetically modified such that photon absorption capability is enhanced as compared to a corresponding wildtype photoautotrophic organism. The photoautotrophic organism can be genetically modified to have enhanced biomass production capability as compared to a corresponding wildtype photoautotrophic organism.

In some embodiments of the invention, a method of producing biomass comprises culturing a photoautotrophic organism in a medium contained in a bioreactor operably linked to a light source under conditions such that more than about 0.3, 1, 3, 5, 10, 15, 30, 50, 75, 125, 175 or 200 grams of biomass per liter of medium. Optionally, more than about 0.3, 1, 3, 5, 10, 15, 30, 50, 75, 125, 175 or 200 grams of biomass per liter of medium is produced in less than about 50, 40, 30, 20, 15, or 10 hours.

In other embodiments of the invention, a method of culturing a photoautotrophic organism comprises introducing said photoautotrophic organism to a bioreactor, wherein the bioreactor comprises a container operably linked to a light source that is configured to emit at least one or more wavelengths of light reaching said container, and wherein the at least one or more wavelengths of light are adjustable based on a growth profile of said photoautotrophic organism, and operating said bioreactor to provide at least one or more wavelengths of light that support growth of said photoautotrophic organism. Where desired, the one or more wavelengths correspond to the one or more peak absorption wavelengths of the at least one light absorption pigment of said photoautotrophic organism.

The present invention provides for a method of culturing a photoautotrophic organism comprising a) introducing said photoautotrophic organism to a bioreactor, wherein the bioreactor comprises a container operably linked to a light source that is configured to emit at least one or more wavelengths of light reaching said container; b) determining a growth profile of said phototrophic organism; and c) adjusting the at least one or more wavelengths of light based results of step b). Where desired, the at least one or more wavelengths are adjusted to maximize growth rate. Optionally, at least one or more wavelengths can be adjusted to maximize growth of the photoautotrophic organism relative to energy supplied to the light source. In some embodiments of the invention, the method can further comprise adjusting the light source to emit pulses of light.

In other embodiments of the invention, a method of culturing a photoautotrophic organism comprises a) introducing said photoautotrophic organism to a bioreactor, wherein the bioreactor comprises a container operably linked to a light source that is configured to emit at least one or more wavelengths of light reaching said container; b) measuring a biomass production by said phototrophic organism; and c) adjusting the at least one or more wavelengths of light based the results of step b). The at least one or more wavelengths can be adjusted to maximize production of a biomass. Where desired, the method can further comprise controlling the light source to emit pulses of light.

In some embodiments of the invention, a method for harvesting a photoautotrophic organism from a bioreactor comprises activating a movable unit mounted in the bioreactor, wherein said activating includes translating the movable unit horizontally or vertically along a length of said bioreactor, concentrating said photoautotrophic organism on one side of the movable unit, and harvesting said photoautotrophic organism on one side of the movable unit through a harvest port extending from said length of said bioreactor to collect a solution of concentrated photoautotrophic organism. The movable unit can comprise a perforated material or a mesh. The concentrated photoautotrophic organism can be suspended in a growth media. Optionally, the method can further comprise transferring the solution of concentrated photoautotrophic organism from the bioreactor to a holding tank and separating the photoautotrophic organism from the growth media to form a solution of further concentrated photoautotrophic organism. In other embodiments of the invention, the method can further comprise loading a hydraulic ram press with the solution of further concentrated photoautotrophic organism; operating the hydraulic ram press to extract oil; and collecting the oil. The method for harvesting a photoautotrophic organism can further comprise collecting the growth media separated from the photoautotrophic organism and returning the growth media to the bioreactor. Optionally, the photoautotrophic organism is algae.

The present invention provides for a method of culturing a photoautotrophic organism, comprising producing electrical energy from a renewable energy source; utilizing said electrical energy to power a light source, wherein said light source emits at least one or more wavelengths of light that reach a bioreactor to support growth of said photoautotrophic organism in said bioreactor. Where desired, the energy converter is selected from the group consisting of a solar panel, a wind turbine, a combustion device, a steam turbine, and a dam. Optionally, the renewable energy source is solar energy. The renewable energy source can be selected from the group consisting of wind energy, hydroelectric energy, biomass energy, and thermal energy. Producing electrical energy can be carbon neutral.

In some embodiments of the invention, a method of culturing a photoautotrophic organism comprises introducing said photoautotrophic organism to a bioreactor, wherein the bioreactor comprises a container comprising a light-receiving element configured to receive solar light for culturing said phototrophic organism during day time; and maintaining growth in the absence of said solar light using an artificial light source.

In other embodiments of the invention, a method comprises culturing a photoautotrophic organism in a bioreactor operably linked to a light source that emits light for growth of said photoautotrophic organism, wherein the bioreactor comprises a container including a reflective element to substantially preclude light loss from or through said container.

The invention provides for a method for producing energy using a manufacturing plant comprising growing a photoautotrophic organism in a bioreactor for producing a biomass; using a power plant for producing electricity and carbon dioxide from said biomass; and supplying said electricity and carbon dioxide to said bioreactor for production of said biomass.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

FIG. 1 is an illustration of an exemplary container for growth of a photoautotrophic organism.

FIG. 2 is a graph showing examples of light absorbing pigments and corresponding relative absorptions at varying wavelengths of light.

FIG. 3 is an illustration of one embodiment of a bioreactor powered by solar energy and/or wind energy.

FIG. 4 is an illustration of an exemplary bioreactor powered by solar energy and combustion of biomass.

FIG. 5 is a diagram showing one embodiment of a bioreactor powered by renewable energy and combustion of biomass.

FIG. 6 depicts an exemplary bioreactor powered by solar light. The bioreactor is mounted on a skid.

FIG. 7 is an illustration showing flow of energy for production of biomass products.

FIG. 8 is a plot showing a spectrum of light emitted by a white light emitting diode.

FIG. 9 is a specification sheet for an exemplary light emitting diode.

FIG. 10 is a specification sheet for an exemplary light emitting diode.

FIG. 11 is a diagram of an exemplary non-imaging optical device.

FIG. 12 is a diagram of an exemplary light distributing element.

FIG. 13 is a diagram of one embodiment of a light distributing element.

FIG. 14 is a diagram of an exemplary light distributing element connected to a computer.

FIG. 15 is a diagram of an exemplary light distributing element comprising one or more plates of an optically transparent material.

FIG. 16 is a cross-sectional view of one embodiment of a light distributing element comprising an array of light sources.

FIG. 17 is a cross-sectional view of an exemplary light distributing element comprising a bifacial array of light sources.

FIG. 18 is a diagram of an exemplary bioreactor with an external light source.

FIG. 19 is a diagram of one embodiment of a bioreactor with multiple containers and multiple external light sources.

FIG. 20 is a diagram of an exemplary bioreactor with an external light source.

FIG. 21 is a diagram of one embodiment of a bioreactor configured for use with an external light source.

FIG. 22 is a diagram of an exemplary bioreactor with a light conducting channel controlled by a computer.

FIG. 23 is a diagram of an exemplary bioreactor with a container having reflective walls.

FIG. 24 is a diagram of one embodiment of a frame of a movable unit.

FIG. 25 is a diagram of one embodiment of a cleaning element.

FIG. 26 is a diagram of an exemplary light conducting channel and a cleaning element positioned around the light conducting channel.

FIG. 27 is a diagram of an exemplary gas sparger.

FIG. 28 is a diagram showing exemplary factors that can be controlled during growth of a photoautotrophic organism.

FIG. 29 is an exemplary diagram showing monitor and control points on a bioreactor.

FIG. 30 is a diagram of one embodiment of an assembly of light conducting channels comprising a frame for supporting the light conducting channels and other components for growth of a photoautotrophic organism.

FIG. 31 is a diagram of an exemplary assembly of light conducting channels without a frame.

FIG. 32 is an exemplary diagram showing steps of bioreactor process cycle.

FIG. 33 is a diagram of one embodiment of a subarray of bioreactors.

FIG. 34 is a diagram of one embodiment of an array of bioreactors controlled by a computer .

FIG. 35 is an exemplary plot of a growth profile for a photoautotrophic organism.

FIG. 36 is an exemplary plot of two growth profiles for a photoautotrophic organism.

FIG. 37 is an illustration of one embodiment of a manufacturing plant for producing energy by combusting biomass produced in a bioreactor.

FIG. 38 is a diagram showing an exemplary bioreactor ready to be filled with a growth media.

FIG. 39 is a diagram showing an exemplary bioreactor with a movable unit of a biomass collector in a mid-level position.

FIG. 40 is a diagram showing an exemplary bioreactor with a movable unit of a biomass collector in a position ready for growth of a photoautotrophic organism.

FIG. 41 is a diagram showing an exemplary bioreactor with a photoautotrophic organism inoculated in the growth media.

FIG. 42 is an exemplary diagram showing harvest of a photoautotrophic organism by movement of a movable unit of a biomass collector.

FIG. 43 is an exemplary diagram showing a bioreactor with concentrated photoautotrophic organism on one side of a movable unit.

FIG. 44 is an exemplary diagram showing collection of a solution of concentrated photoautotrophic organism through a harvest port.

FIG. 45 is an exemplary diagram showing extraction of biomass products using a biomass extractor.

FIG. 46 is an exemplary diagram showing separation of aqueous and non-aqueous biomass products in a separation tank.

FIG. 47 is a diagram showing one embodiment of a bioreactor with a biomass collector.

FIG. 48 is an illustration of one embodiment of a bioreactor with a biomass collector, a container that can be used for distribution of light from an array of light sources, and a reflector.

FIG. 49 is an illustration of an exemplary bioreactor with multiple light conducting channels and a biomass collector.

FIG. 50 is an illustration of an exemplary bioreactor with a biomass collector and a container that can be used for distribution of light from an array of light sources.

FIG. 51 is a diagram showing one embodiment of an array of bioreactors for producing biomass.

FIG. 52 is a diagram showing one embodiment of an array bioreactors with a collection tank for producing biomass.

### DETAILED DESCRIPTION OF THE INVENTION

The invention provides for devices and methods for using photoautotrophic organisms to produce biomass and/or industrial chemicals that can be used as a renewable energy source. The devices of the invention include, but are not limited to, biomass extraction devices and biomass combustion plants, as well as bioreactors and the components therein for growing and harvesting one or more photoautotrophic organisms. Among others, components of the bioreactor can include but are not limited to (1) devices for collecting energy and delivering energy in the form of light to the bioreactor, devices for production, transmission, and distribution of light, (2) devices for monitoring and controlling growth conditions, and (3) devices for harvesting one or more photoautotrophic organisms from the bioreactor. Also provided by the present invention include without limitation (1) methods for delivering light and other resources to a bioreactor, (2) methods of controlling growth parameters for efficient growth of one or more photoautotrophic organisms, (3) methods for harvesting one or more photoautotrophic organisms, (4) methods for biomass extraction, and (5) methods for producing renewable energy from biomass.

While preferable embodiments of the invention have been shown and described herein, it will be obvious to those skilled in the art that such embodiments are provided by way of example only. The devices and/or methods of the invention can be employed individually or combined with other devices, methods, and/or systems in manners know to those skilled in the arts for efficient and high production of biomass using photoautotrophic organisms.

### Devices of the Invention

A bioreactor can comprise one or more components to support the growth of one or more photoautotrophic organisms. These components can include apparatuses to contain a growth media, devices to collect solar, thermal, hydroelectric, or wind energy for supplying electricity to the bioreactor, devices for production, transmission, and distribution of light, devices for monitoring and controlling growth conditions, and devices for harvesting one or more photoautotrophic organisms from the bioreactor.

In one aspect of the invention, a bioreactor comprises a container for culturing a photoautotrophic organism, said photoautotrophic organism having at least one light absorption pigment, the at least one light absorption pigment having one or more peak absorption wavelengths; and a light source configured to emit one or more wavelengths of light reaching said container, wherein the one or more wavelengths of light are adjustable based on a growth profile of said photoautotrophic organism.

In some embodiments of the invention, a bioreactor comprises a container including a light-receiving element configured to receive solar light for culturing a photoautotrophic organism; and a light conducting channel operably linked to said light-receiving element, wherein said light conducting channel having a surface area that transmits light from at least about 50% of surface area of said channel.

In another embodiment of the invention, a bioreactor comprises a container for culturing a photoautotrophic organism, wherein said container comprises a movable unit mounted therein, wherein said movable unit is adapted to translate horizontally or vertically along a length of said container, wherein said translation concentrates said photoautotrophic organism on one side of said movable unit; and a harvest port extending from said length of said container to collect said concentrated photoautotrophic organism.

The present invention provides for a bioreactor comprising a container for culturing a photoautotrophic organism, a light source configured to emit one or more wavelengths of light that reaches said container to support growth of said photoautotrophic organism, and an energy converter for production of electrical energy from a renewable energy source, wherein said energy converter is operably linked to said light source.

In some embodiments of the invention, a bioreactor comprises a container comprising a light-receiving element configured to receive solar light for culturing a photoautotrophic organism during day time; and a light source configured to emit one or more wavelengths of light reaching said container to maintain growth of said photoautotrophic organism in the absence of said solar light.

In other embodiments of the invention, a bioreactor comprises a container for culturing a photoautotrophic organism, said container comprising a reflective element to substantially preclude light loss from or due to said container; and a light source configured to emit one or more wavelengths of light reaching said container to support growth of said photoautotrophic organism.

In another aspect of the invention, an array of light sources comprises one or more light sources configured to emit at least one or more wavelengths of light, wherein light emission from said array of light sources is configured to be adjustable to match one or more peak absorption wavelengths of a light absorption pigment that is contained in a photoautotrophic organism.

The invention provides for a manufacturing plant for production of biomass and electric energy, comprising a bioreactor for production of biomass, said biomass comprising a photoautotrophic organism and a container; and a power plant operably linked to said bioreactor, wherein said power plant converts said biomass to electricity and carbon dioxide, wherein said carbon dioxide is supplied to said bioreactor for production of said biomass.

### Containers

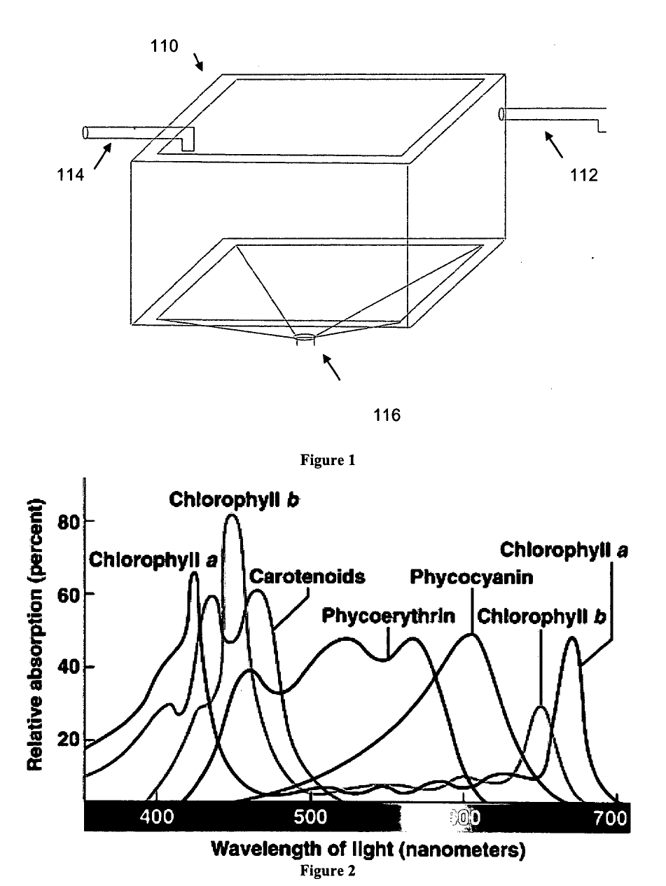

The bioreactor can include a container of any type that allows for substantially water-tight containment of a growth media. The container can be used for culturing a photoautotrophic organism. In some embodiments of the invention, the container can be a preexisting container like a shipping container, a storage bladder, or a pool. Alternatively, the container can be fabricated from materials like glass, concrete, polymers, metal, semi-conductor, or any combination thereof. A metal that can be used includes steel, aluminum, iron, copper, bronze, or any combination thereof. The form of the container can be substantially box-like, cylinder-like, spherical, or any other shape. The walls of the container can be flexible, rigid, or any combination thereof. The container can be such that a positive or negative pressure can be maintained relative to surrounding conditions. An example of a container is shown in FIG. 1. FIG. 1 shows a tank or vessel (110) with a water and nutrient input port (114), a drain (116), and a harvest port (112). The drain can be used for removal of a culture of photoautotrophic organisms, removal of dead photoautotrophic organisms, or for removal of any other materials and liquids that accumulate at the bottom of the tank or vessel. The tank or vessel can have one or more harvest ports. The one or more harvest ports can be used for removal of materials and liquids from the tank or vessel. The tank can have a top, middle, and bottom position along the height of the tank. In some embodiments of the invention, the harvest port extends from the side of the tank near the top of the tank, the middle of the tank, or the bottom of the tank. The one or more harvest ports can be used to remove liquids or materials from the tank.

Interior walls of the container can be coated with or constructed of a reflective material. The reflective material can allow for reduced loss of light due to transmission of light through the walls of the container or absorption of light energy into the walls of the container. Reduction in amount of light lost through and/or due to the walls of the container can increase the amount of light available for use by the one or more photosynthetic organisms for growing in the bioreactor. An example of a reflective material can include an aluminum sheet, a polymer, silver, or an aluminum oxide. Commercial products that can be used as a reflective material include Everbrite® by Alcoa or Mylar® by Dupont. The reflective material can be chosen to have high reflectivity for the one or more wavelengths of light used for supporting growth of the one or more photoautotrophic organisms. The reflective material can reflect up to 98% of incident light or more. The reflective material may be smooth, textured, or shaped as an option to further the function of light distribution.

In other embodiments of the invention, walls of the container are optically transparent and exterior surfaces of the walls are coated with a reflective material. The Walls can be such that light that is incident on an interior wall of the container is transmitted through the walls and to the reflective material, where then the light is reflected back into the container.

The size of the bioreactor can be any size suitable for economically feasible growth of a photoautotrophic organism. In some embodiments of the invention, the bioreactor can be approximately 20 feet wide by 40 feet long by 20 feet deep. In other embodiments of the invention, the bioreactor is 1 cubic feet in volume. The dimensions of the bioreactor can be chosen such that the components of the bioreactor can allow for efficient production of biomass.

### Organisms

Any type of photoautotrophic organisms can be grown using the devices and/or methods described herein. In some embodiments of the invention, one, two, three, or more photoautotrophic organisms can be grown in the bioreactor concurrently, sequentially, or any combination thereof. In other embodiments of the invention, a photoautotrophic organism can be grown with a photochemotrophic organism or a heterotrophic organism concurrently, sequentially, or any combination thereof. Heterotrophic organisms that can be grown concurrently or sequentially with photoautotrophic organisms can include fish, like tilapia, cyprinids, or sea bass, or crab.

Photoautotrophic organisms can be broken down into aquatic and terrestrial photoautotrophic organisms. Examples of terrestrial photoautotrophic organisms that can be grown include miscanthus, switchgrass, pine, corn, and soybean. Examples of aquatic photoautotrophic organisms include chiarella vulgaris, haematococcus, stichochoccus, bacillariophyta (golden algae), cyanophyceae (blue green algae), chlorophytes (green algae), chlorella, botryococcus braunii, cyanobacteria, prymnesiophytes, coccolithophorads, neochloris oleoabundans, scenedesmus dimorphus, atelopus dimorphus, euglena gracilis, dunaliella, dunaliella salina, dunaliella tertiolecta, diatoms, bacillariophyta, chlorophyceae, phaeodactylum tricomutum, stigmatophytes, dictyochophytes, and pelagophytes.

The photosynthetic or photoautotrophic organism can be chosen based on robustness, improved growth, or improved biomass production. Improved growth can be measured on a time basis or on a basis of nutrients supplied, such as light or carbon dioxide. In some embodiments of the invention, the photosynthetic organism can be chosen based on one or more light absorbing pigments belonging to the photosynthetic organism. A light absorbing pigment can include carotenoid, carotene, alpha-carotene, beta-carotene, phycobilin, phycocyanin, phycoerythrin, allophycocyanin, fucoxanthin, xanthophylls, luteol, fucoxanthol, violaxanthol, chlorophyll A, chlorophyll B, chlorophyll c1, chlorophyll c2, or chlorophyll d. A light absorbing pigment can preferentially absorb one or more wavelengths. As shown in FIG. 2, chlorophyll A preferentially absorbs light at approximately 430 nm and 670 nm and chlorophyll B preferentially absorbs light at approximately 470 nm and 650 nm. Likewise, wavelengths that are preferentially absorbed by carotenoids, phycoerythrin, and phycocyanin are shown.

Photosynthetic organisms can contain one or more light absorbing pigments. For example, all plant, algae and cyanobacteria contain chlorophyll A, cyanobacteria contain phycobilin, green algae contain chlorophyll B, red algae contain phycoerythrin, brown algae and diatoms contain fucoxanthin. These light absorbing pigments can be used to drive photosynthesis in the photosynthetic organisms.

The photoautotrophic organism can be modified for robustness, improved growth, or improved biomass production. The improved growth can be determined based on a growth profile. The growth profile can be represented by measurements of dry cell weight of the photoautotrophic organism per volume or of optical density of said photoautotrophic organism over a period of time.

Improved growth can be compared to a corresponding wildtype photoautotrophic organism. Improved growth or biomass production can be measured on a time basis or on a basis of nutrients supplied, such as light, carbon dioxide, or growth media. The photoautotrophic organism can be modified by performing random mutagenesis, rational mutagenesis, directed evolution, or any combination thereof. Directed evolution can include creating a library of organism variants and screening variants for a desired property. In other embodiments of the invention, the photoautotrophic organism can be modified using metabolic engineering.

The photoautotrophic organisms used in the present invention, such as algae, can be pre-adapted and pre-conditioned to specific environmental and operating conditions used for growth in the bioreactors of the invention. The productivity and long-term reliability of algae utilized in a bioreactor for producing biomass or biomass products can be enhanced by utilizing algal strains and species that are native or otherwise well suited to conditions and localities in which the bioreactor will be utilized.

As is known in the art (see, for example, Morita, M., Y. Watanabe, and H. Saiki, “Instruction of Microalgal Biomass Production for Practically Higher Photosynthetic Performance Using a Photobioreactor.” Trans IchemE. Vol 79, Part C, September 2001.), algal cultures that have been exposed to and allowed to proliferate under certain sets of conditions can become better adapted for long term growth and productivity under similar conditions. The present invention can utilize photoautotrophic organisms that have been or can be reproducibly and predictably pre-conditioned and pre-adapted to increase their long term viability and productivity under a particular expected set of operating conditions. The photoautotrophic organisms can be pre-conditioned and pre-adapted to prevent bioreactors inoculated with such algal species from having other undesirable algal strains contaminate and dominate the algal culture in the bioreactor over time.

Desirable strains of algae can be difficult to maintain in a bioreactor that is not scrupulously sterilized and maintained in a condition that is sealed from the external environment. The reason for this is that the algal strains being utilized in such bioreactors may not be well adapted or optimized for the conditions of use, and other, endemic algal strains in the atmosphere are more suitably conditioned for the local environment, such that if they have the ability to contaminate the bioreactor, they will tend to predominate and eventually displace the desired algae species. Such phenomena can be mitigated and/or eliminated by using the pre-conditioned and pre-adapted photoautotrophic organisms. Use of algae strains can not only increase productivity and longevity of algal cultures in real bioreactor systems, thereby reducing capital and operating costs, but also can reduce operating costs by eliminating the need to sterilize and environmentally isolate the bioreactor system prior to and during operation, respectively.

Typically, commercially available algal cultures are adapted to be grown under ordinary laboratory conditions. Accordingly, such commercially available algal cultures may not be well-suited to be grown under one or more conditions of light exposure, gas supply, temperature, etc. to which algae would be expected to be exposed to for biomass production. For example, most commercially available algal cultures are conditioned for growth at relatively low light levels, such as 150 micro Einstein per meter squared per second (150 μEm⁻²s⁻¹). Exposure of such cultures to sunlight in bioreactor may expose the organisms to light intensities of 2,500 μEm⁻²s⁻¹ or greater and may cause substantial photoinhibition, rendering such cultures unable to survive and/or grow adequately, and, therefore, unable to successfully compete with deleterious native species that may infiltrate the bioreactor. Accordingly, one aspect of the invention is to utilize photoautotrophic organisms that have been pre-conditioned and pre-adapted to light of an intensity and duration expected to be experienced a bioreactor of the invention.

In addition, the bioreactors, in certain embodiments, may be configured and operated to subject the algae to relatively high frequency photomodulation cycles or intermittent light. While such high-frequency photomodulation or intermittent light can be beneficial for the growth of the algae, unadapted and unconditioned algal strains may not be well adapted to and ideally suited for growing under such conditions. Accordingly, in certain embodiments, the algal strains can be pre-adapted and pre-conditioned for growth under conditions of high-frequency photomodulation or intermittent delivery of light described herein. Similarly, many components found in typical flue gases, which may be removed by the bioreactors of the current invention in certain embodiments, may be lethally toxic to and/or can substantially inhibit growth of nonadapted algal strains at concentrations that may be found in flue gas. For example, the concentration of CO₂, NOₓ, SOₓ, and heavy metals such as Hg in flue gases may be toxic or deleterious to many unadapted algal strains. The present invention provides for utilizing photoautotrophic organisms that have been pre-conditioned and pre-adapted for exposure to such toxic or deleterious gases.

### Energy Supply

The bioreactors of the invention can utilize energy for a variety of purposes. These purposes include supplying light, allowing for heating or cooling, compressing gases, or powering electronics. The bioreactors of the invention can be powered using electricity obtained from conventional sources or from renewable energy sources. Use of renewable energy sources such as solar energy, wind energy, biomass energy, thermal energy, or hydroelectric energy can require an energy converter for conversion of the renewable energy sources to electricity. Hydroelectric energy can comprise any energy that can be produced using hydrodynamic force. For example hydroelectric energy can utilize tidal change, waves, water passageways, or water height change. Other devices that can be implemented when using renewable energy sources can include an energy storing device, an inverter, or an energy conditioning device.

A device that can convert a renewable energy source to electricity, herein also called an energy converter, can include a solar panel, a solar thermal device, a wind turbine, a combustion device, a steam turbine, a dam, a water wheel, or any combination thereof. Other devices for energy conversion known to those skilled in the arts can be an energy converter. The device for producing electrical energy can be carbon neutral, meaning that no additional carbons are produced while producing electrical energy from a renewable energy source. The calculation of carbon production can include the growth and/or production of the renewable energy source.

Solar panels, in particular, can be useful for producing electrical energy from solar energy. A solar panel can comprise one or more types of pn junctions for conversion of solar energy to electrical energy. Examples of pn junction types include silicon, GaAs, AlGaAs, cupric indium diselenide, CdTe, and other semiconductor materials known to those skilled in the arts. The solar cell can comprise one or more types of pn junctions for collection of one or more ranges of light wavelengths. A solar panel can be a paint-on solar panel known to those skilled in the arts. A solar cell, or any solar device described herein, can be mounted on a device for tracking a solar light source. A light concentrator described herein or of any other type can be used in conjunction with any device utilizing solar energy.

Alternatively, solar energy can be converted to electrical energy using a solar thermionic system. The solar thermionic system can concentrate solar energy and convert the solar energy to heat. The heat can be used to power a turbine (e.g. a steam turbine) to generate electricity. Solar thermionic devices are described in U.S. Pat. No. 6,302,100 and U.S. Pat. No. 3,467,840, both of which are herein incorporated by reference.

A combustion device can include a chamber for converting biomass or any material from chemical energy into combustion products, such as heat, carbon dioxide, and water. The heat can be used to convert a liquid to a gas, for example water to steam, and used to power a turbine for generation of electricity. The carbon dioxide and water can be used as nutrients for growth of a photoautotrophic organism in a bioreactor described herein. The biomass used in the combustion device can be biomass produced by growth of a photoautotrophic organism grown in a bioreactor described herein.

A wind turbine can be used to convert wind energy to electrical energy. A wind turbine can be a vertical axis wind turbine or a horizontal axis wind turbine. The wind turbine can be any wind turbine known to those skilled in the arts.

The energy storing device, which can include a battery, can be used to store excess energy. This can allow for the bioreactor to be powered in the absence of a renewable energy source or under conditions where the amount of power generated from a renewable energy source is variable. The battery can be a lithium ion battery, a lead-based battery, or any other type of rechargeable battery known to those skilled in the arts. This can be useful, for example, for operating a solar powered bioreactor during the night when solar energy is not available or for operating a wind powered bioreactor when the amount of wind energy available is variable.

The inverter can be used to convert an alternating current power supply to a direct current power supply and vice-versa. This can alleviate problems encountered when energy converters produce an alternating current power supply when a direct current power supply is needed by a load or vice-versa.

The energy conditioning device can be used to normalize electrical power from an energy converter and/or from a battery. The energy conditioning device can stabilize electrical power to be supplied to a bioreactor such that the electrical equipment in the bioreactor is not supplied an incorrect amount of power.

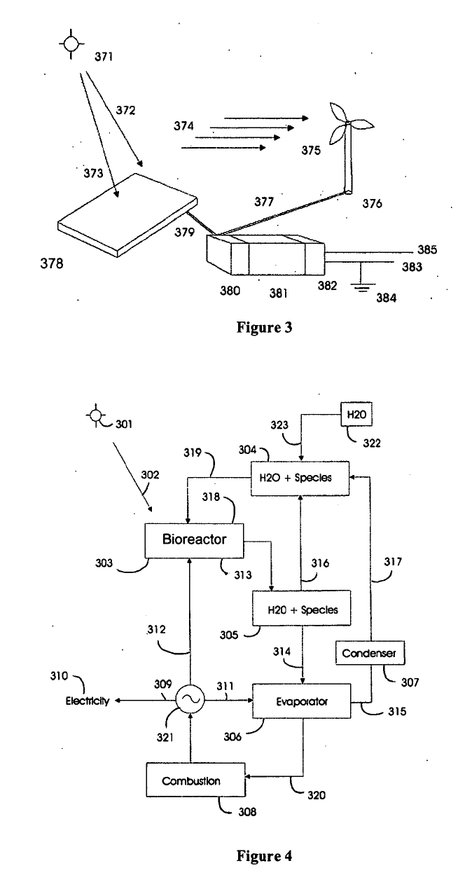

One embodiment of an electrical energy supply system is shown in FIG. 3. The electrical energy supply system can include a solar panel (378), a wind turbine (375), an energy conditioning device (380), a battery (381), and an inverter (382). The solar panel can accept solar energy produced by the sun (371) through photons that are emitted from the sun (372) and impinge (373) on the solar panel (378). The solar panel can convert solar energy to electrical energy, which is transferred to the energy conditioning device through a first electrical connection (379). The wind turbine can convert wind energy (374) into electrical energy. The wind turbine can be supported on a tower or by other deployment hardware (376). Electrical energy produced by the wind turbine can be transferred to the energy conditioning device through a second electrical connection (377). The energy conditioning device can normalize electrical energy from the wind turbine and the solar panel prior to transferring the energy to a battery. The battery can store energy for supplying energy on demand. Energy can be converted into alternating current using the inverter. Current from the inverter can be supplied to a load using a negative lead (383) and a positive lead (385). The negative lead can be connected to a ground (384).

Another embodiment of an electrical energy supply system is shown in FIG. 4. The bioreactor (303) comprises a photovoltaic panel, a container for growing a photoautotrophic organism, and an array of lights. The bioreactor is powered by solar rays (302) from the sun (301). The photoautotrophic organism can be mixed with water and any other nutrients in a pre-mixing vat (304) to form a growth media. Water (322) from a source is fed into the pre-mixing vat through a controlled pipeline (323). The photoautotrophic organism and growth media are fed to the bioreactor through an intake pipeline (319). Reactant gases, such as oxygen, and any feedstock gas unabsorbed by the growth media within the bioreactor is vented from the specific invention through an exit pipe (318). The exit pipe can contain monitoring elements. The monitoring elements can be used for monitoring gases exiting the bioreactor such as oxygen, carbon dioxide, nitrogen oxides, and sulfur oxides.

Photoautotrophic organisms grown in the bioreactor and the growth media can exit the bioreactor through an exit pipeline (313) into a secondary holding vat (305). A return pipeline (316) can be used to transfer material from the secondary holding vat to the pre-mixing vat.

Photoautotrophic organisms grown in the bioreactor and the growth media in the secondary holding vat are transported through a pipeline (314) to an evaporator (306) for drying. Liquid in the evaporator can be evaporated or siphoned off. Evaporated liquid can be condensed in a condenser (307) and then sent to the pre-mixing vat. Solid biomass and other biomass products can be fed to a combustion device (308) for production of heat by combustion.

A heat-engine (321), such as a steam engine, or other thermal engines known in the art, employing Rankine or other thermodynamic cycles, can utilize the heat for production of electrical energy. Unused heat (311) from the combustion and electricity generation process is transported by means known in the art to the evaporator (306) to drive evaporation of liquid media. Electricity (310) is produced from the heat engine (321) and can be used to power the bioreactor and other components or elements described herein. Excess electricity can be stored or transported elsewhere. Combustion of biomass in the combustion device results in CO₂ and other flue gases (312) that are delivered to the heat engine and then can be delivered to the bioreactor for growth of photoautotrophic organisms.

### Light Sources

Supply of light is a component for maintaining growth of a photoautotrophic organism. Light delivery systems of the present invention can include devices to make use of artificial light or natural light. Artificial light can include light produced by any electrically powered light source. Natural light can include solar light, any type of light naturally found in an environment, or any type of light that is not artificial light. Utilization of both artificial and natural light can allow for a robust bioreactor that can function in a variety of environments under a range of conditions. A light source can be configured for the growth of a photoautotrophic organism. The light source can be configured to emit one or more wavelengths, to emit pulses of light, to emit light intermittently, or to emit an intensity of light based on growth of a photoautotrophic organism or production of a biomass product by the photoautotrophic organism.

An electrically powered light source can include a light emitting diode, a gas discharge bulb, a laser, an incandescent bulb, a high pressure sodium bulb, or a metal halide bulb. An example of a gas discharge bulb can be a fluorescent light bulb. A light source can be configured or chosen to emit a range of light wavelengths, to emit a range of light intensities, and/or to convert electricity to light at a range of efficiencies. As discussed in “Light-emitting Diodes”, 2nd Edition, E. Fred Schubert, different light sources can have varying luminous efficiencies. For example, light sources can have the following efficiencies: tungsten filament—15 to 20 lm/watt, quartz halogen—20 to 25 lm/watt, fluorescent—50 to 80 lm/watt, mercury vapor—50 to 60 lm/watt, metal halide—80 to 125 lm/watt, high pressure sodium—100 to 140 lm/watt, organic LED—1,300 to 130,000 lm/watt, and III-V LED—13 million to 130 million lm/watt. As such, some light sources can be more efficient than others at converting electricity to light.

The energy of a photon is a function of its frequency and Planck's constant. This relationship is given by E=hν=h*c/λ, where Planck's constant, h=6.626×10⁻³⁴ joules-second, ν is the frequency of the photon, the speed of light, c=3×10⁸ m/s, and λ is the wavelength of the light.

A light source can have an efficiency in converting energy to photons, expressed as a percentage based on energy output in the form of photons divided by the energy input in the form of joules of electricity. The efficiency can be no less than 5%, 10%, 20%, 30%, 40%, 50%, 60%, 70%, 80%, 90%, 95%, or 99%.

A light source can produce a desired range of light wavelengths, or spectrum of light. The range of wavelengths can span 0.5 nm, 1 nm, 3 nm, 5 nm, 10 nm, 20 nm, 50 nm, 100 nm, 200 nm, 500 nm or more.

A light source can be configured to have an emission of light that is continuous or intermittent. For intermittent emission of light, the light emission can be regular or irregular. For regular light emission, the frequency can be from 0.05 to 2000 Hz, 5 to 1000 Hz, or 10 to 500 Hz. Light can be emitted 1-95%, 5-90%, or 10-80% of the total time. The intensity of the light over time can be described by a light intensity waveform. The light intensity waveform can be triangular, saw-tooth, square, sinusoidal, or any other desired shape.

In addition to other types of light sources, a light emitting diode is an example of a light source that can efficiently convert electricity to light and produce light of particular wavelengths. Light emitting diodes can also provide other advantages such as being low-cost, long-lasting, and durable. A light emitting diode can be chosen based on a range of wavelengths produced, such that the range of wavelengths produced better match wavelengths needed for growing a photoautotrophic organism. The photoautotrophic organism can have one or more peak absorption wavelengths. The one or more wavelengths emitted by the light source can correspond to the one or more peak absorption wavelengths of at least one light absorption pigment of the photoautotrophic organism.

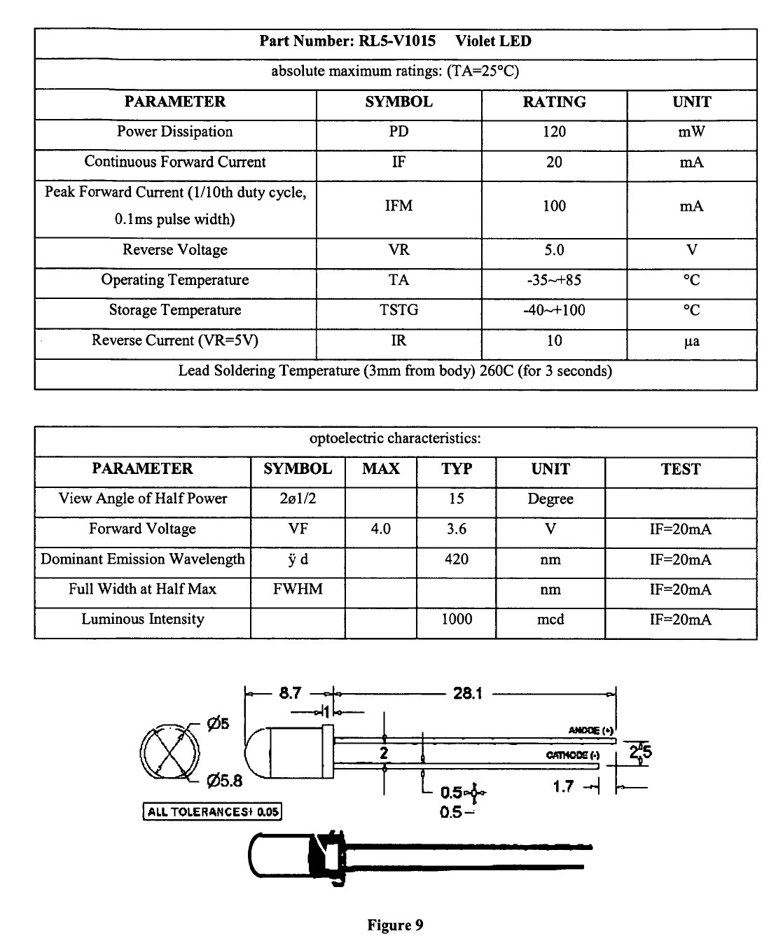

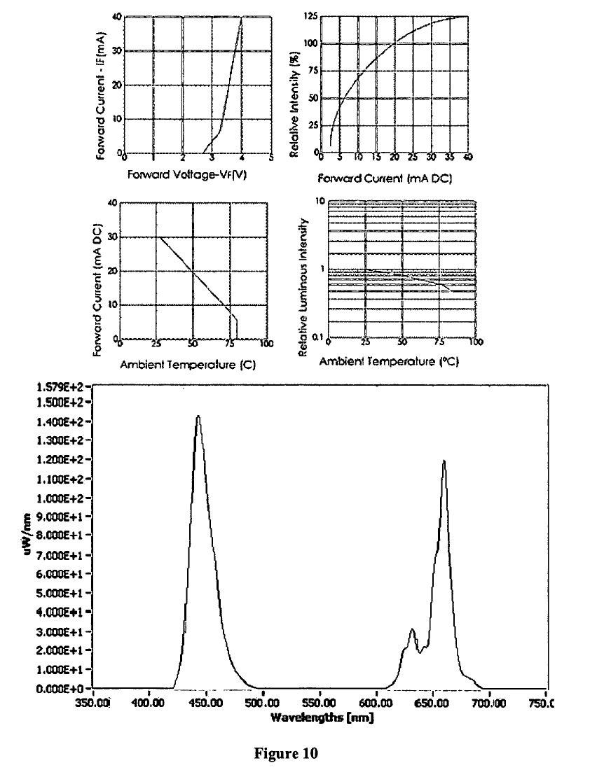

A light emitting diode can be specified in terms of a wattage requirement, a range of light output, a dominant emission wavelength, a luminous intensity, an operating temperature, or a size. For example, specifications for a light emitting diode are shown in FIG. 9 and FIG. 10. A range of light output for the light emitting diode shown in FIG. 9 and FIG. 10 is shown in a graph of FIG. 10 that plots μW/min against wavelengths. The graph shows that the light emitting diode can have two peak light emission wavelengths. The two peak light emission wavelengths can be approximately 445 nm and 655 nm. The light emitting diode can emit a range of light that spans a range of wavelengths, for example, approximately 425 nm to 500 nm and approximately 610 nm to 690 nm. Light emitting diodes can be purchased from any light emitting diode supplier.

In another embodiment of the invention, a light emitting diode with two peak light emission wavelengths can be used to supply light to a bioreactor for growth of a photoautotrophic organism. The two peak light emission wavelengths can be approximately 445 nm and 655 nm. The photoautotrophic organism can be an organism comprising chlorophyll A, where chlorophyll A has peak light adsorption wavelengths that are approximately 460 nm and 670 nm.

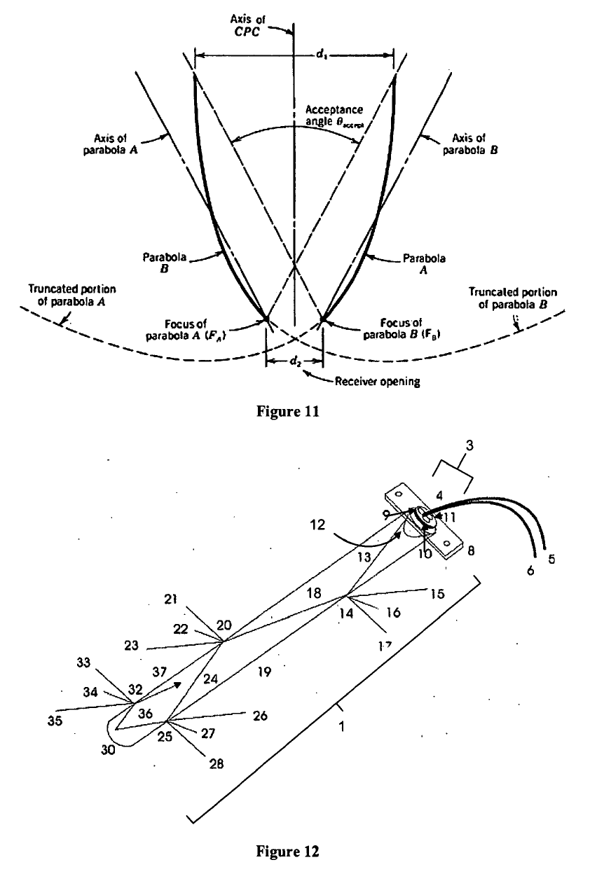

In some embodiments of the invention, solar light can be used to as a sole light source for providing light to a bioreactor or solar light can be used in combination with an artificial light source. A light-receiving element can be used to collect solar light and direct solar light to the bioreactor. The light-receiving element can comprise a glass, polymer, or a metal. The light-receiving element can comprise any light concentrator that can collect light directed to an area and concentrate the light into a smaller amount of area. In some embodiments of the invention, a light-receiving element comprises one or more of the following: an optical fiber cable, a light collecting dish, a window, a parabolic trough concentrator, or a non-imaging optical device. For example, the non-imaging optical device can be a compound parabolic concentrator. As shown in FIG. 11, the compound parabolic concentrator can have an acceptance angle, an axis of a parabola, a parabola, a receiver opening, and an axis of the compound parabolic concentrator. The compound parabolic concentrator can have a wide acceptance angle. The acceptance angle can be greater than 10, 20, 30, 40, 50, 60, 70, 80, 90, 100, 120, 130, 150, or 170 degrees. The acceptance angle can be less than 10, 20, 30, 40, 50, 60, 70, 80, 90, 100, 120, 140, 160, or 180 degrees. The compound parabolic concentrator can also have a concentration ratio. The concentration ratio can be determined by the following equation.

Concentration Ratio=1/[sin(Acceptance Angle/2)]

The concentration ratio of the compound parabolic concentrator can be between approximately 0.5 to 6, between approximately 1.3 to 5, or approximately 2. The compound parabolic concentrator can be adjusted to track the movement of the sun continuously, every hour, every day, every week, or every month.

### Transmission and Distribution of Light

One common problem with growing photoautotrophic organisms is transmission and distribution of light for optimal growth of the photoautotrophic organisms. Growth of photoautotrophic organisms to high quantities can hinder the transmission and distribution of light by physical occlusion of a light source, preventing photosynthesis from occurring in the photoautotrophic organisms. This problem can be circumvented by improved delivery of light within the bioreactor. The improved delivery of light can be through an increase in surface area capable of emitting light into a growth media. Delivery of light to and within the bioreactor can comprise a light transmitter and a light distributor. The light transmitter and light distributor can be used to transmit light from a light source placed inside or outside the bioreactor and distribute the light over a greater amount of area. The light transmitter and light distributor can be a light conducting channel.

The light conducting channel can be such that a wide range of wavelengths of light can be transmitted within the light conducting channel or distributed from the light conducting channel without substantial loss of light due to absorption by the light conducting channel. The light conducting channel can be constructed such that a portion of the light conducting channel can distribute light within the bioreactor. The portion of the light conducting channel that can distribute light can be at least 30%, 40%, 50%, 60%, 70%, or 80% of an exterior surface area of the channel. The light conducting channel can be constructed to emit a substantially uniform intensity of light from the exterior surface area used to distribute light. As shown in FIG. 12, the light conducting channel can utilize a convex surface (12) to facilitate uniform distribution of light.

The light conducting channel can comprise one or more materials. The one or more materials can comprise a glass, a plastic, a polymer, or a reflective element. In some embodiments of the invention, an optical fiber cable can be used to transmit light. A particular type of glass that can be used, for example, can be Pyrex®. In other embodiments of the invention, the light conducting channel can be a glass plate. The light conducting channel can be constructed such that it can withstand hydrostatic pressure or any other forces once the bioreactor is filled with a growth media. For example, the light conducting channel can be constructed using a high strength material. The light conducting channel can be solid or hollow. A hollow light conducting channel can be filled with air or a liquid, or can maintain a vacuum. As shown in FIG. 12 and FIG. 15, the shape of the light conducting channel can be substantially rod-like or box-like. As shown in FIG. 12, the reflective element can be positioned at an end of the light conducting channel (30) or at any other locations in the light conducting channel to increase light distribution. The reflective element can be a mirror or any other reflective material mentioned previously. As shown in FIG. 12 and FIG. 15, light sources and/or arrays of light sources can be placed on the exterior or interior of the light conducting channel. In some embodiments of the invention, the container of a bioreactor can be a light conducting channel and a reflective material can be placed on the exterior of the light conducting channel to contain light within the bioreactor.

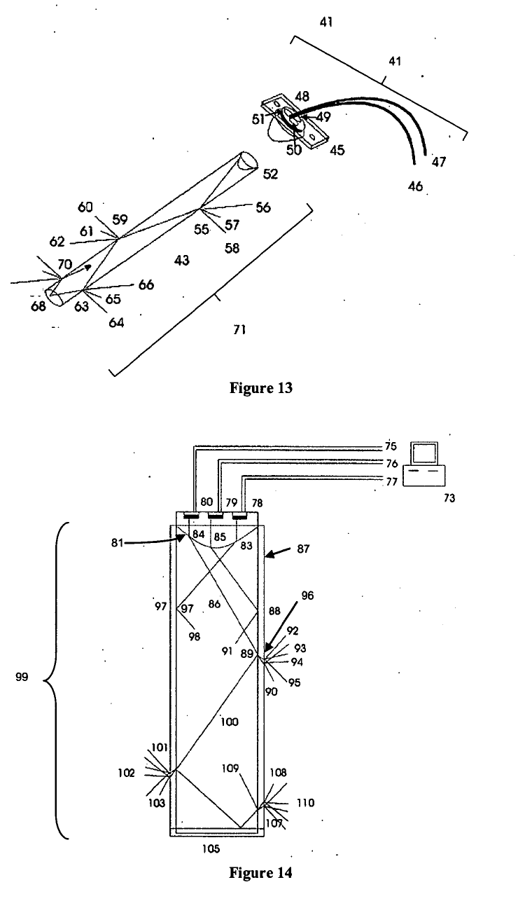

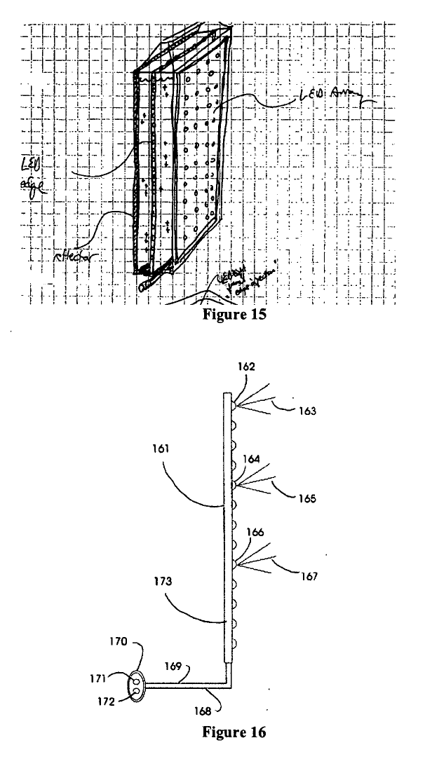

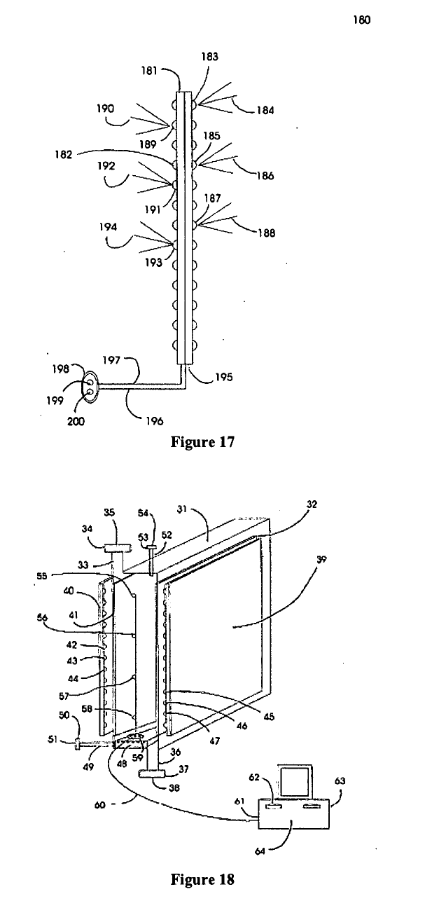

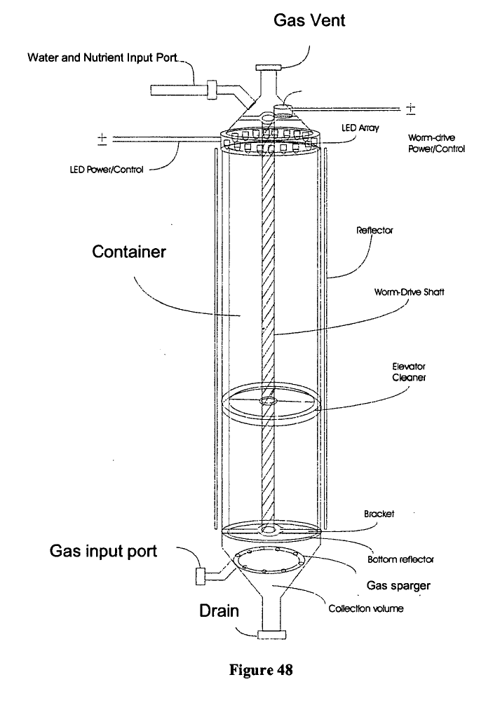

Examples of light transmitters and light distributors are shown in FIG. 12, FIG. 13, FIG. 14, FIG. 15, FIG. 16, FIG. 17, and FIG. 48.

As shown in FIG. 12, a light distributing element (1) can comprise an array of lights (3) and a light conducting channel (1). The array of lights can comprise multiple light sources (9, 10, 11) energized by an electrical current delivered by two leads (5, 6) connected to a wiring harness (4) that electrically connects the two leads to the multiple light sources. The multiple light sources (9, 10, 11) can be the same or different materials and can emit one or more wavelengths that are the same or different. The multiple light sources and wiring harness can be supported by a mounting flange (8). The mounting flange can be mechanically connected to the light conducting channel.

The light conducting channel shown in FIG. 12 can comprise a convex surface (12) that is formed by grinding, casting, or forming of optical materials using methods known by those skilled in the arts, such that photons emitted by the light emitting diode array elements (9, 10, 11) can be directed throughout the light conducting channel. The photons, upon impinging the convex threshold, can either be internally reflected (18), will be emitted as photons that exit the light conducting channel (15, 16, 17) or be absorbed by the light conducting channel.

The photons that are internally reflected (18) can impinge again on a surface of the light conducting channel (20), and can be reflected (24), absorbed, or emitted (21, 22, 24). Photons (24) that are not absorbed or emitted can impinge (25) again on a surface of the light conducting channel (25), and can be reflected (36), absorbed, or emitted (26, 27, 28). Those photons (36) that are not absorbed or emitted continue until they impinge on a bottom surface (30). The bottom surface can be coated on an internal side with a reflective material. The reflective material can be a material such as Everbrite® by Alcoa or Mylar® by Dupont. Alternatively, the reflective material can be deposited using vapor deposition using any method known by those skilled in the arts. The photons that are reflected at the bottom surface can impinge on the light conducting channel (32) and be either reflected, absorbed, or emitted (33, 34, 35).

The light conducting channel can be filled with a gas, liquid, or a solid. Alternatively, the light conducting channel interior can consist of a vacuum.

FIG. 13 shows an alternate view of the light distributing element shown in FIG. 12. The light distributing element can comprise an array of lights (41) and a light conducting channel (71). The array of lights can comprise multiple light emitting sources (49, 50, 51), a wiring harness (48) for connecting wire leads to the multiple light emitting sources, a mounting flange (45) to support the array of lights, and two wire leads (46, 47) for supplying current to the multiple light emitting sources. The light conducting channel can comprise a convex surface (52) for accepting photons emitted by the multiple light emitting sources and directing the photons toward throughout the light conducting channel. The photons can impinge on the light conducting channel (55, 59, 65, 70) and be either reflected, absorbed, or emitted (56, 57, 58, 60, 61, 62, 63, 64, 65). The light conducting channel can have a bottom surface (68) that is coated with a reflective material. The reflective material can allow for photons impinging on the bottom surface to be reflected.

The light distributing elements shown in FIG. 12 and FIG. 13 can be used to transmit and distribute light in a bioreactor for growth of a photoautotrophic organism. A bioreactor can comprise one or more of these light distributing elements, such that the distribution of light throughout the bioreactor is substantially uniform. The arrangement of the light distributing elements can be regular or irregular.

FIG. 14 shows a cross-sectional view of an alternate embodiment of a light distributing element. The light distributing element can comprise multiple light sources (78, 79, 80) that emit one or more photons (83, 84, 85) at one or more wavelengths. The multiple light sources can be controlled by a computer (73) through electrical connections (75, 76, 77). The one or more photons can be impinge on a convex surface (81) that can direct the photons through a light conducting channel (99). The photons can travel through the interior of the light conducting channel (86) and impinge on an interior surface of the light conducting channel (89, 97). Upon impinging on the interior surface of the light conducting channel, the photons can be reflected (98, 100), absorbed, or emitted (92, 93, 94, 95, 101, 102, 103, 107, 108, 110). The photons that are emitted from the light conducting channel travel through an optically transparent material that comprises the walls of the light conducting channel (87). Angles of light travel and emission of light can be predicted by Snell's law.

FIG. 15 shows an alternate embodiment of light distributing elements that can be utilized in a bioreactor. The light distributing elements can comprise one or more plates of an optically transparent material. The optically transparent material can be a polymer or a glass. The optically transparent material can be solid or hollow. Hollow materials can be filled with a liquid, a gas, or a vacuum. The one or more plates of optically transparent material can have one or more light sources positioned on one or more exterior edges of the plates. In one embodiment, one or more optically transparent plates are placed in a substantially rectangular-box bioreactor. One or more light sources are positioned along a top edge of the one or more optically transparent plates. One or more walls of the bioreactor that are parallel to the optically transparent plates are coated with a reflective material. The photons emitted by the one or more light sources are directed to the optically transparent plates such that photons travel through the optically transparent plates and are distributed throughout the bioreactor by emission of photons from the optically transparent plates and into a surrounding environment.

FIG. 16 shows a cross-sectional view of one embodiment of a light distributing element that can be used to deliver light to a bioreactor. The light distributing can comprise an array of light sources (161) with one or more light sources. The light sources (162, 164, 166) can be the same or different. In some embodiments of the invention, the light sources are light emitting diodes. The light sources can emit one or more wavelengths of light (163, 166, 167). The one or more wavelengths of light can correspond to one or more absorptions wavelengths of a light absorbing pigment.

The array of light sources can be mounted on a stand (173), suspended, or attached to another object by any means known to those skilled in the arts. The array of lights can be powered by one or more electrical leads (169, 168). The electrical leads can be connected to one or more terminals (171, 172) and a terminal connector (170). The array of lights can be controlled by a computer and can be physically and electrically independent of the bioreactor.

FIG. 17 shows an embodiment of a light distributing element with an array of light sources that can be used to deliver light from both sides of the array of light sources. The light distributing element can be built such that the light distributing element occupies a minimal amount of space. The light distributing element can comprise a bifacial array of light sources (181), a first set of one or more light sources (183, 185, 187) placed on a first side of the array of light sources and a second set of one or more light sources (182, 189, 191, 193) placed on a second side of the array of light sources. The first and second sides can be opposite sides of the array of light sources. The one or more light sources can emit one or more wavelengths of light (184, 186, 188, 190, 192, 194) that are the same or different. Power can be supplied to the array of light sources by connection of a power bus to the array (195). The power can be supplied through electrical connections (196, 197) that are connected to terminals (199 and 200) and a terminal connector (198).

FIG. 48 shows an embodiment of a bioreactor comprising a container that is a light conducting channel. The container can be constructed of an optically transparent material such that light reaching the container is distributed throughout the bioreactor. The optically transparent material can comprise acrylic or glass. As shown in FIG. 48, the container can be coated or wrapped with a reflective material. The reflective material can reflect light incident on the inside surface of the exterior of the bioreactor back into the bioreactor. Reflective material can also be placed on the bottom or top sides of the bioreactor such that light on the interior of the reactor that is incident on the top or bottom of the bioreactor is reflected back into the bioreactor. As shown in FIG. 48, one or more light sources can be placed at edge of the container such that light emitted from the one or more light sources enters a wall of the container and is distributed throughout the bioreactor. The one or more light sources can be a LED array. The LED array can be controlled through electrical connections to a controller, such as a computer.

The bioreactor shown in FIG. 48 also comprises an elevator cleaner mounted on a worm drive shaft. The elevator cleaner can move along a length of the bioreactor substantially parallel to the worm drive shaft by rotation of the worm drive shaft. The elevator cleaner can comprise one or more cleaning elements for cleaning the interior walls of the container. The cleaning element can be a brush-like material. The worm drive shaft can be mounted on a bracket. Other features of the bioreactor shown in FIG. 48 include a gas input port for supply of gases, such as carbon dioxide, a gas vent for releasing excess gas, such as oxygen and/or carbon dioxide, a water and nutrient input port, a gas sparger, a collection volume for collecting dead or damaged photoautotrophic organisms, and a drain. The dead or damaged photoautotrophic organisms that settle at the bottom of the bioreactor can be exported through the drain.

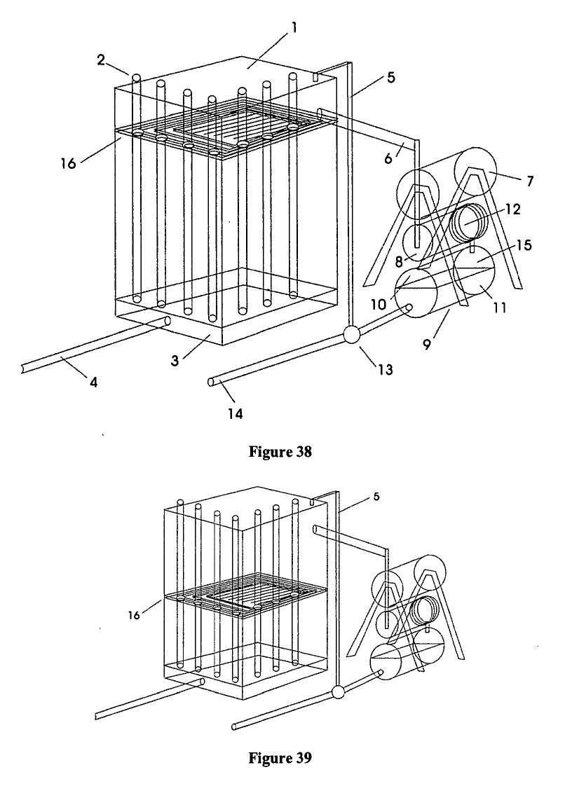

As shown in FIG. 38 one or more light conducting channels (2) can be placed in a bioreactor. The light conducting channels can occupy up to 5%, 10%, 20%, 40%, 50, or 60% of the total volume inside the bioreactor container. Arrangement of the one or more light conducting channels can be adjusted based on a desired level and/or pattern of light distribution.

A combination of one or more light sources can be used as an array of light sources, allowing for production of a wide range of wavelengths and control over the one or more wavelengths of light emitted by the light emitting array. The range of light produced by the one or more light sources can be from 300 nm to 800 nm. In some embodiments of the invention, certain ranges of light between 300 nm and 800 nm are not produced or are produced at significantly lower amount than other ranges of light between 300 nm and 800 nm. The ranges of light that can be produced at a significantly lower amount than other ranges of light can be between about 530 and 610 nm. The one or more wavelengths of light emitted by the array of light sources can be chosen to correspond to one or more wavelengths that are photosynthetically active wavelengths. Photosynthetically active wavelengths, also referred to as photosynthetically available radiation, can correspond to wavelengths of light that can be absorbed by one or more light absorption pigments. A light absorption pigments can be any light absorption pigment found in a photoautotrophic organism. Photosynthetically active wavelengths are shown in FIG. 2.

In some embodiments of the invention, an array of light sources can comprise a light conducting channel, described herein, for transmitting photons emitted from the light emitting sources. The light sources can be mounted on the inside or outside of the light conducting channel. The light sources can be configured to emit pulses of light.

The devices of the present invention include an assembly comprising devices for light production, transmission, and distribution. The devices for light production, transmission, and distribution are those devices described previously. The assembly can be used independent of a bioreactor, used for growth of a photoautotrophic organism, or used as a replacement part for the bioreactors described herein. In some embodiments of the invention, the assembly can be immersed in a pond or other body of water that can be used for growth of a photoautotrophic organism.

### Biomass Collector

The bioreactors of the present invention can comprise a container with a biomass collector for harvesting a photoautotrophic organism grown inside the bioreactor. The biomass collector can comprise a movable unit that can be mounted inside the container and configured to translate horizontally or vertically along a length of the container. The movable units can be configured to move from a top portion of the bioreactor to a bottom portion of the bioreactor or from the bottom portion of the bioreactor to the top portion of the bioreactor. In some embodiments of the invention, the bioreactor can comprise more than one movable unit, such that the movable units can be translated in a direction toward each other to concentrate the photoautotrophic organism.

The movable unit can be mechanically attached to a worm drive screw or cabling to allow for movement of the movable unit along a length of the container. The movable unit can be configured to translate such that only about 3 to 50%, 5% to 40%, or 10 to 30% of the volume of the container is on one side of the movable unit.

The movable unit can comprise a perforated material or a mesh that can allow for a growth media to pass through the movable unit as the movable unit translates. The perforated material or mesh can be configured to selectively capture a photoautotrophic organism as the movable unit translates based on size.

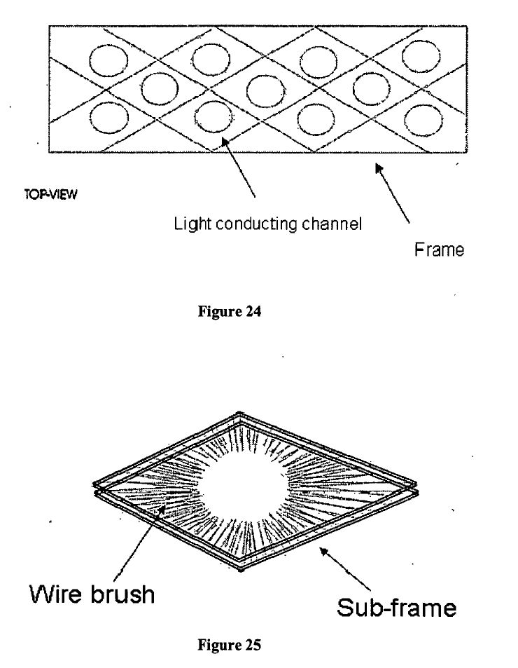

The movable unit can comprise a frame. As shown in FIG. 24, the frame can be constructed to surround one or more light conducting channels arranged in the bioreactor. The frame can comprise one or more wires. In other embodiments of the invention, the movable unit can comprise a cleaning element that comprises a wire brush or a wire mesh and a sub-frame supported by the frame. The cleaning element can be used to clean one or more light conducting channels that are arranged on the inside of the bioreactor and are used for transmission and distribution of light. As shown in FIG. 25, the cleaning element can comprise a wire mesh or wire brush that is supported by a sub-frame.

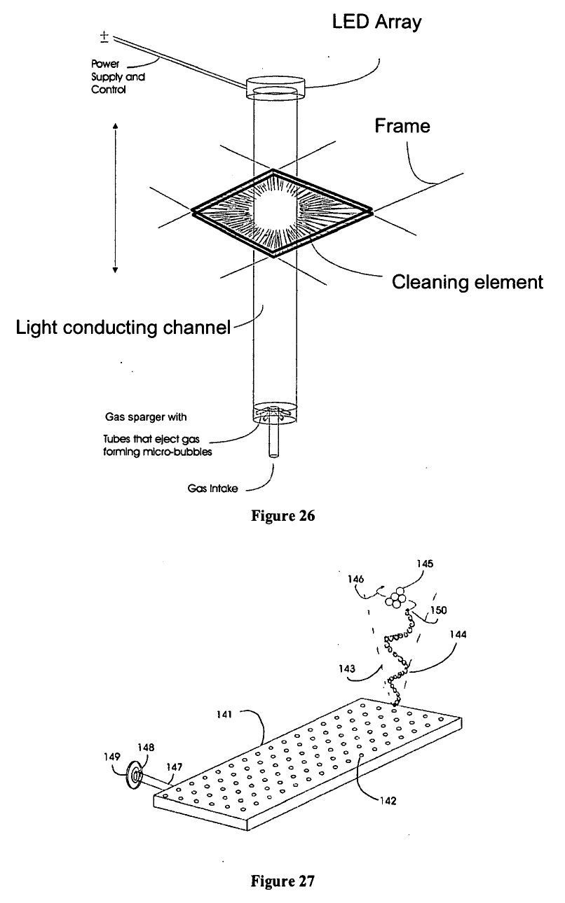

As shown in FIG. 26, a cleaning element can be placed around a light conducting channel for cleaning the light conducting channel. The cleaning element can be supported by a frame that is movable. In some embodiments of the invention, the light conducting channel can be supplied light by an LED array for producing one or more wavelengths of light that is distributed by the light conducting channel. As shown in FIG. 26, the LED array can be powered and controlled through electrical connections. In other embodiments of the invention, the light conducting channel can be mechanically connected to a gas sparger. The gas sparger can comprise one or more tubes that eject gas and form micro-bubbles. A gas intake can supply gas to the gas sparger.

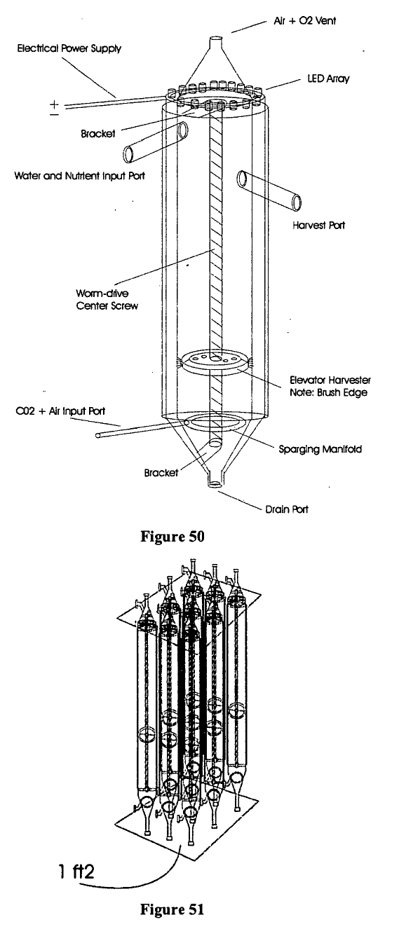

Alternatively, the cleaning element can be placed on the inside of a light conducting channel. An example of a cleaning element placed on the inside of a light conducting channel is seen in FIG. 50, where the cleaning element is depicted on an elevator harvester as a brush edge.

In some embodiments of the invention, the bioreactor can comprise a harvest port extending from a length of the container. The harvest port can be used to collect a concentrated photoautotrophic organism. The concentrated photoautotrophic organism can be suspended in a liquid.

### Gas Supply

The present invention provides for a bioreactor comprising a sparger for delivery of gas to the bioreactor.

The gas sparger can be comprised of materials like polymers, plastics, metals, or glass. In some embodiments of the invention, the gas sparger is injection molded or rotomolded. In other embodiments of the invention, the gas sparger is manufactured from stainless steel.

Gases supplied to the bioreactor can include carbon dioxide, nitrogen, oxygen, air, or helium. Carbon dioxide is commonly used as a carbon source for growth of photoautotrophic organisms. Carbon dioxide can be obtained from industrial suppliers, industrial flue gases, combustion sources, or the atmosphere. In some embodiments of the invention, carbon dioxide is obtained from organisms performing fermentation. Alternatively, carbon dioxide can be obtained from the combustion of methane produced by digestion or fermentation of biomass, such as agricultural manure. The introduction of carbon dioxide into the bioreactor can be performed using the gas sparger described herein.

As shown in FIG. 27, the gas sparger (141) can have one or more holes (142). The holes can be less than approximately 0.01, 0.05, 0.1, 0.25, 0.5, or 1 cm in diameter. Gas, air, CO2, or other feed gases can be fed to the sparger through conduit (147) that is connected to a controllable valve (148). Feedstock gas or gases are fed at gas intake port (149) and controlled by opening and closing of the controllable valve (148). When the bioreactor contains a growth media, the one or more holes can allow for the formation of small gas bubbles or micro-bubbles when gas is delivered to the bioreactor. Reduction in size of the gas bubbles can increase the surface area of the gas-liquid interface, therefore increasing the adsorption of the gas into the growth media. Pressurization of gas in the gas sparger can cause the release of a stream of very small micro-bubbles. These micro-bubbles emanate from the pressurized sparger (141) through small holes (142) and produce a cone of micro-turbulence (143) that travel up from the holes through the growth media. These micro-bubbles (144) act on photoautotrophic organisms in the growth media (145) and induce tumbling or mixing (146, 150) that improve exposure of the photoautotrophic organism to light and other nutrients.

As shown in FIG. 26, the gas sparger can be placed near or beneath a light conducting channel. Placement of the gas sparger near the light conducting channel can improve delivery of a gas to a photoautotrophic organisms due to a tendency for photoautotrophic to migrate toward a light source. In some embodiments of the invention, the gas sparger can be placed such that the bubbles from the gas sparger increase mixing or turbulence of a growth media inside the bioreactor.

Unused gas supplied to the bioreactor or gas produced by the photoautotrophic organism can exit the bioreactor through one or more exit ports. A pressure relive valve or any other control valve can control pressure inside the bioreactor and a rate of gas exit from the bioreactor. Gas exiting the bioreactor, for example unused carbon dioxide, oxygen produced by the photoautotrophic organism, or any other gas, can be collected and used in other processes. In some embodiments of the invention, a compressor can be used for the collection of gas exiting the bioreactor.

### Nutrient Supply

In some embodiments of the invention, the bioreactor comprises devices for delivery of liquid nutrients to the bioreactor. Liquid nutrients can include water and water containing phosphorus, magnesium, nitrates, nitrites, phosphates, silica, salts, and/or trace elements. Trace elements can include molybdenum, iron, cobalt, copper, zinc, manganese, lead, cadmium, sulfur, calcium or nickel. The devices for delivery of liquid nutrients can include one or more ports placed on the bioreactor. The ports can be located on the top, side, or bottom of the bioreactor. The ports can be connected to hosing, tubing, or piping that can be used for transport of liquid materials using any means known to those skilled in the arts. The hosing, tubing or piping can be connected inline with a pump for providing hydrodynamic force to drive liquid flow.

Growth media compositions, nutrients, etc. required or suitable for use in maintaining a growing algae or other photoautotrophic organisms are well known in the art. Potentially, a wide variety of growth media can be utilized in various forms for various embodiments of the present invention, as would be understood by those of ordinary skill in the art. Potentially appropriate growth media components and nutrients are, for example, discussed in detail in: Rogers, L. J. and Gallon J. R. “Biochemistry of the Algae and Cyanobacteria,” Clarendon Press Oxford, 1988; Burlew, John S. “Algal Culture: From Laboratory to Pilot Plant.” Carnegie Institution of Washington Publication 600. Washington, D.C., 1961 (hereinafter “Burlew 1961”); and Round, F. E. The Biology of the Algae. St Martin's Press, New York, 1965; each incorporated herein by reference).

### Control Devices

The bioreactor can comprise devices for controlling temperature. The devices for controlling temperature can be heating devices or cooling devices. The devices for heating can include a heat exchanger or an electrical resistor. The heat exchanger can use any liquid or gas, such as water, glycerol, oil, or steam. The gas or liquid can be heated using solar energy or thermal energy generated from combustion. The heat exchanger or electrical resistor can be placed around the bioreactor in a cooling jacket, or be placed on the interior of the bioreactor. The devices for cooling can include a heat exchanger or a Peltier circuit placed on the exterior or interior of the bioreactor. The heat exchanger can be supplied with cooling water or be connected to a compressor containing one or more refrigerants. Alternatively, cooling or heating of a bioreactor can be facilitated by sprinkling of water to the exterior of the bioreactor.

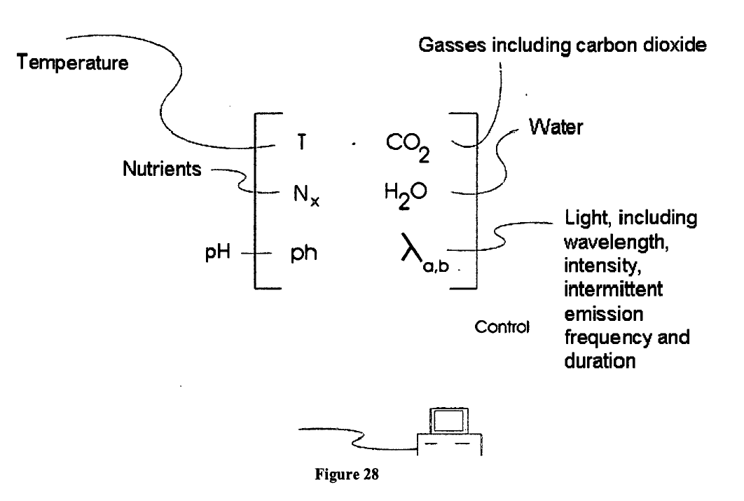

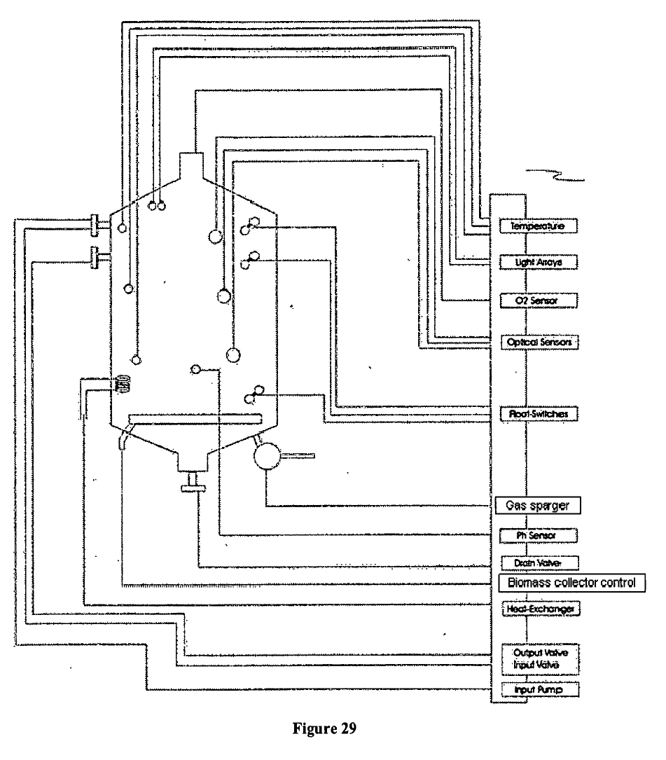

The bioreactors described herein can be used for the growth of a photoautotrophic organism. Growth of a photoautotrophic organism can be facilitated by monitoring and controlling one or more growth conditions. Growth conditions that can be monitored and controlled can be selected from the group consisting of pH, light conditions, gas conditions, temperature, pressure, volume, biomass, and biomass products concentrations. The growth conditions can be monitored and controlled using any devices known by those skilled in the arts. In some embodiments of the invention, the growth conditions are monitored and controlled in real-time. The growth conditions can be monitored and controlled without user-intervention. FIG. 28 is a depiction of several growth conditions that can be monitored and controlled during the growth of a photoautotrophic organism.

As shown in FIG. 29, a bioreactor described herein can include one or more components for monitoring and controlling the bioreactor. These components can include a temperature sensor, a light array, an oxygen sensor, an optical sensor, a float switch, a gas sparger, a pH sensor, a drain valve, a biomass collector control, a heat exchanger, an output valve, an input valve, and an input pump. The bioreactor can be controlled based on a growth profile of a photoautotrophic organism growing in the bioreactor. The components for monitoring and controlling the bioreactor can include a computer for collecting data from the bioreactor and interacting with control points of the bioreactor. The computer can allow for remote control of the bioreactor. The computer can be electrically connected to one or more components for monitoring or controlling the bioreactor. Electrical connections between the one or more components for monitoring or controlling the bioreactor and the computer can be RS232 cables, Ethernet cables, serial cables, parallel cables, or fiber optics cables. In other embodiments of the invention, the computer is connected wirelessly to the components for monitoring and controlling the bioreactor. Examples of computer systems for monitoring and controlling the bioreactor can include Supervisory Control and Data Acquisition (SCADA) systems.

The components for monitoring the bioreactor can include measuring components for monitoring conditions selected from the group consisting of pH, light conditions, gas conditions, temperature, pressure, volume, biomass, or biomass products. The measuring components can be any device known by those skilled in the arts. In particular, the measuring components for measuring biomass can include a light source and a light sensor. The measuring components for measuring biomass products can include a mass spectrometer, a gas chromatograph, a liquid chromatograph, or any combination thereof. The measuring components for monitoring the bioreactor allow for in-line and/or real-time measurement of bioreactor conditions. The components for monitoring the bioreactor can be used to determine a growth profile of a photoautotrophic organism growing in the bioreactor.

The components for controlling the bioreactor can include controller components for controlling bioreactor parameters selected from the group consisting of gas supply, heating or cooling, pressure, light supply, or nutrient supply. The controller components for controlling gas supply, heating or cooling, pressure, light supply, or nutrient supply can be any device known by those skilled in the arts. Light supply can be characterized by one or more wavelengths of emitted light, an intensity of emitted light, and a pattern of intermittent light emission. The controller components for controlling light emission can allow for control over the one or more wavelengths of emitted light, the intensity of emitted light, and the pattern of intermittent light emission.

The controller components can include hardware and/or software for implementing the control over the bioreactor parameters. For example, the controller components can include electronics that allow communication of electrical signals between the computer and the controller components for controlling the bioreactor. Communication can be established using pulse width modulation, voltage signals, or any other type of electrical, optical, or wireless communication.

In some embodiments of the invention, devices can be implemented for controlling the emission of light from the light sources. These devices can include resistors, transistors, capacitors, inductors, or any combination thereof. These components can allow for emission of a controllable intensity of light or emission of a pattern of intermittent light.

### Additional Bioreactor Components

The bioreactor of the present invention can include a baffle, a pressure relief valve, a condenser, a viewing window, or any other component included in a bioreactor known by those skilled in the arts. The baffle can be positioned along the interior walls of the bioreactor or can be positioned to baffle water that is filling the tank.

### Biomass Extraction

One aspect of the invention provides for devices used to process biomass from one or more photoautotrophic organisms. The devices used to process biomass can include a device for extracting biomass products from biomass. The biomass can be one or more photoautotrophic organisms.

A holding tank can be used to hold biomass comprising one or more photoautotrophic organisms grown in a bioreactor and suspended in liquid. The holding tank can allow for separation of the photoautotrophic organism from the liquid using gravity. Liquid separated from the photoautotrophic organism can be recovered. The recovered liquid can be water and/or other growth nutrients and returned to the bioreactor tor growth of photoautotrophic organisms. The holding tank can have an interior volume of from about 1-100,000 ft³, 100-60,000 ft³, 1,000-40,000 ft³, or 3,000-10,000 ft³. The holding tank can be comprised of steel, aluminum, glass, or polymer materials.

In some embodiments of the invention, the device used to extract biomass products from biomass is any device capable compressing the biomass and allowing for biomass products to be mechanically extracted from the biomass. In some embodiments of the invention, the device used to extract biomass products is a hydraulic press, a screw, a metal crusher, or a centrifuge. The device for extraction of biomass products from biomass can be similar to a device used to extract oil from cocoa beans or olives.

Alternatively, the device used to extract biomass products from biomass is any device capable utilizing chemical extraction methods to collect biomass products from the biomass. The device for extracting biomass products can be a device similar to one used to chemically extract and/or refine olive oil from olives. The device used to extract biomass products can be an apparatus that utilizes a hexane solvent or a supercritical fluid, such as liquefied CO₂, to extract biomass products from biomass. The devices for chemical extraction can be manufactured from steel or aluminum to withstand high operating pressure requirements and explosion hazard requirements. Components for heating and cooling, such as a heat exchanger, can be implemented for temperature control.

The device used to extract biomass products from biomass can include a combination of mechanical and chemical methods described above. In some embodiments of the invention, extraction of biomass products includes a device for mechanical extraction of biomass products followed by a device for chemical extraction of biomass products.

In some embodiments of the invention, a manufacturing plant can be used for the production of biomass and electrical energy. The system can comprise a bioreactor for growing a photoautotrophic organism and a power plant that is operably linked to the bioreactor for converting biomass or biomass products produced by the photoautotrophic organism to electricity and carbon dioxide. The carbon dioxide can be supplied to the bioreactor for production of biomass by the photoautotrophic organism. The bioreactor can comprise a light source configured to emit one or more wavelengths of light reaching the bioreactor, where emission of light is adjustable based on a growth profile on the photoautotrophic organism.

One embodiment of the invention is shown in FIG. 5. FIG. 5 shows a flow chart of energy and mass-transfer involving a system for producing electricity and straight vegetable oil comprising a bioreactor (344), a renewable electricity energy generator (343), a biomass extractor (353), a combustion device (364), and a turbine (361). The bioreactor (344) receives energy from a renewable electricity energy generator (343), such as a solar panel or a wind turbine. The sun (341) can provide solar energy (342) to a solar panel.

The bioreactor (344) receives CO₂, flue gas, NO_{X}, SO_{X}, or other gases (345) from combustion of organic material (364). Growth media, comprising water and nutrients, and a photoautotrophic organism in aqueous solution are stored in a pre-mixing vat (347). Input valves controlled can be controlled by a computer and are used to transport water, nutrients, and a photoautotrophic organism to the bioreactor through an intake pipe (348). The pre-mixing vat can be fed water through a pipe (350) from a water source (349). Photoautotrophic organisms grown in the bioreactor and growth media can be delivered to a secondary vat (352) through a pipe (346). The photoautotrophic organism in growth media are fed a biomass extractor (353). The biomass extractor can be a hard press used in the food preparation industry. The photoautotrophic organism and growth media can be subjected to high pressure such that straight vegetable oil (355) and press cake can be produced. The straight vegetable oil (355) is harvested using any means known by those skilled in the arts (354) and can be used as a biodiesel feedstock or other applications. Press cake and all non-oil materials can be delivered to an evaporator (362) for collection of excess water through a pipe, conveyor belt, any combination thereof, or any other transferring means (356). Water in liquid form and gas form is transferred to a condenser (358) through pipes (357) for condensation and then delivery to the pre-mixing vat through a pipe (359). Press cake can be dried in the evaporator and then removed using any means known to those skilled in the arts (363). Dried press cake can be transferred to a combustion device (364) to be burned. Oxygen is taken from the air and combusted with press cake. Heat can be delivered to a turbine (361) for electricity generation. Waste heat, or rejected heat from the turbine is directed to the evaporator.

### Methods of the Invention

The methods of the present invention can allow for production of biomass or biomass product by growth of a photoautotrophic organism in a bioreactor. The methods include utilizing renewable energy sources for powering the bioreactors and other electrical components, supplying light, utilizing systems for monitoring and controlling conditions of the bioreactor, and utilizing biomass collectors. The photoautotrophic organism can be any photoautotrophic organism described herein.

In some embodiments of the invention, a method for producing biomass comprises culturing a photoautotrophic organism in a medium contained in a bioreactor operably linked to a light source that emits photons to support growth of the photoautotrophic organism, wherein the light source is configured to yield a biomass production efficiency at no less than about 100, 10, 1, 0.1, or 0.01 milligrams of said biomass per kJ of energy that is supplied to the light source.

In other embodiments of the invention, a method of producing biomass comprises culturing a photoautotrophic organism in a medium contained in a bioreactor operably linked to a light source under conditions such that more than about 0.3, 1, 3, 5, 10, 15, 30, 50, 75, 125, 175 or 200 grams of biomass per liter of medium is produced.

The methods of the invention provide for a method of culturing a photoautotrophic organism comprising (a) introducing said photoautotrophic organism to a bioreactor, wherein the bioreactor comprises a container operably linked to a light source that is configured to emit at least one or more wavelengths of light reaching said container, and wherein the at least one or more wavelengths of light are adjustable based on a growth profile of said photoautotrophic organism; and (b) operating said bioreactor to provide at least one or more wavelengths of light that support growth of said photoautotrophic organism.

In some embodiments of the invention, a method of culturing a photoautotrophic organism comprises (a) introducing said photoautotrophic organism to a bioreactor, wherein the bioreactor comprises a container operably linked to a light source that is configured to emit at least one or more wavelengths of light reaching said container; (b) determining a growth profile of said phototrophic organism; and (c) adjusting the at least one or more wavelengths of light based results of step (b).

In other embodiments of the invention, a method of culturing a photoautotrophic organism comprises (a) introducing said photoautotrophic organism to a bioreactor, wherein the bioreactor comprises a container operably linked to a light source that is configured to emit at least one or more wavelengths of light reaching said container; (b) measuring a biomass production by said phototrophic organism; and (c) adjusting the at least one or more wavelengths of light based the results of step (b).

The methods of the invention provide for a method for harvesting a photoautotrophic organism from a bioreactor comprising (a) activating a movable unit mounted in the bioreactor, wherein said activating includes translating the movable unit horizontally or vertically along a length of said bioreactor; (b) concentrating said photoautotrophic organism on one side of the movable unit; and (c) harvesting said photoautotrophic organism on one side of the movable unit through a harvest port extending from said length of said bioreactor to collect said solution of concentrated photoautotrophic organism.

In some embodiments of the invention, a method of culturing a photoautotrophic organism comprises (a) producing electrical energy from a renewable energy source; and (b) utilizing said electrical energy to power a light source, wherein said light source emits at least one or more wavelengths of light that reach a bioreactor to support growth of said photoautotrophic organism in said bioreactor.

In other embodiments of the invention, a method of culturing a photoautotrophic organism comprises (a) introducing said photoautotrophic organism to a bioreactor, wherein the bioreactor comprises a container comprising a light-receiving element configured to receive solar light for culturing said phototrophic organism during day time; and (b) maintaining growth in the absence of said solar light using an artificial light source.

The methods of the invention provide for a method comprising culturing a photoautotrophic organism in a bioreactor operably linked to a light source that emits light for growth of said photoautotrophic organism, wherein the bioreactor comprises a container including a reflective element to substantially preclude light loss from or through said container.

In one aspect of the invention, a method for producing energy using a manufacturing plant comprises (a) using a photoautotrophic organism for producing a biomass; (b) using a power plant for producing electricity and carbon dioxide from said biomass; and (c) supplying said electricity and carbon dioxide to said bioreactor for production of said biomass.

### Methods of Supplying Electrical Energy

A photoautotrophic organism can be grown in a bioreactor that is powered by a renewable energy source. The renewable energy source can be used to power a light source in the bioreactor to produce one or more wavelengths that can reach the bioreactor to support growth of the photoautotrophic organism. The renewable energy source can also be used for compressing air and carbon dioxide or powering pumps, valves, sensors, and biomass extraction devices. The renewable energy source can be energy that is produced by an energy converter selected from the group consisting of a solar panel, a wind turbine, a combustion device, a steam turbine, and a dam. The energy converter can convert a renewable energy source selected from the group consisting of wind energy, hydroelectric energy, biomass energy, and thermal energy to electrical energy. The process of producing electrical energy can be carbon neutral, meaning that no additional carbons are produced while producing electrical energy from a renewable energy source. The calculation of carbon production can include the growth and/or production of the renewable energy source.

In some embodiments of the invention, electrical energy is supplied to the bioreactor using a combination of energy converters. A combination of energy converters can be used to provide a more even production of electrical energy over a variety of environmental conditions. For example, a wind turbine can be combined with a solar panel, such that electricity produced by the solar panel can be used in the presence of solar light, and electricity produced by the wind turbine can be used in the absence of solar light.

Electricity produced by the one or more energy converters and/or one or more renewable energy sources can be used to power a bioreactor or any other electrically powered device. Excess electricity can be stored by an energy storing device, such as a battery. The battery can be any type of battery described herein. The battery can be any rechargeable battery known to those skilled in the arts.

The electrical energy that is produced using one or more energy converters or one or more renewable energy sources can be conditioned prior to being stored by an energy storage device or prior to being supplied to a bioreactor or any other electrically powered device. The energy conditioning device can be used to normalize electrical power from the one or more energy converters and/or from the energy storage device. Use of the energy conditioning device to normalize electrical power can prevent the failure of an electrically powered device by preventing incorrect amounts of voltage and/or current from being delivered to the electrically powered device.

### Methods for Supplying Light

Methods for supplying light to a bioreactor for growth of a photoautotrophic organism can include utilizing solar light, utilizing artificial or electrically powered light sources, or utilizing a combination thereof A solar light source can be combined with an artificial light source to provide for low-cost supply of light and/or supply of light in the absence of solar light. Solar light sources can be utilized with or without concentration of the light source prior to delivery of light to the bioreactor. A solar light source can be concentrated using a light-receiving element.

In some embodiments of the invention, methods for supplying light for growing a photoautotrophic organism can include utilizing a bioreactor with a reflective coating on the interior surfaces of the bioreactor. The reflective surface can reduce the amount of light loss due to or through the materials of the bioreactor.

Light that is delivered to a bioreactor can be controlled. Control of a light source can include controlling one or more wavelengths emitted by the light source or delivered to the bioreactor, controlling intensity of the light emitted by the light source or delivered to the bioreactor, controlling intermittent emission of light or delivery of light to the bioreactor, or controlling the spatial distribution of light.

The one or more wavelengths emitted by the light source or delivered to bioreactor can be wavelengths of light that can be utilized by a photoautotrophic organism. In some embodiments of the invention, the one or more wavelengths are tuned to match one or more light absorbing pigments belonging to one or more photoautotrophic organisms. The one or more wavelengths of light can be tunable by utilizing an array of light sources that comprise one or more light sources. The one or more light sources can emit a range of wavelengths of light.

The one or more wavelengths can be tuned for the production of one or more biomass products. In some embodiments of the invention, a first set of one or more wavelengths can be delivered for production of biomass and a second set of one or more wavelengths can be delivered for production of a biomass product. For example, production of biomass can utilize a set of wavelengths between 310-520 nm and production of a biomass product can be between 600-700 nm.

The amount of the light emitted by the light source or delivered to the bioreactor can be controlled. The amount of light can be between 50 μEm⁻²s⁻¹ to 10,000 μEm⁻²s⁻¹, 100 μEm⁻²s⁻¹ to 7,500 μEm⁻²s⁻¹, or 150 μEm⁻²s⁻¹ to 5,000 μEm⁻²s⁻¹.

The intermittent emission of light or delivery of light to the bioreactor can be controlled. A cycle of intermittent emission of light can also be called a photomodulation cycle. The intermittent emission of light can be regular or irregular. The delivery of light to the bioreactor can be dependent on the emission of light from a light source. For regular intermittent emission of light, the frequency can be from 0.05 to 2000 Hz, 5 to 1000 Hz, or 10 to 500 Hz. Light can be emitted 1-95%, 5-90%, or 10-80% of the total time. The intensity of the light over time can be described by a light intensity waveform. The light intensity waveform can be triangular, saw-tooth, square, sinusoidal, or any other desired shape.

The distribution of light can be controlled by changing the arrangement of one or more light conducting channels used to transmit and distribute light within the bioreactor. The light conducting channels can be spaced closer or further apart or in a uniform or non-uniform pattern.

### Methods for Optimizing Growth Conditions

Growth of a photoautotrophic organism in a bioreactor can be optimized for a number of growth parameters. These growth parameters can include growth rate of the photoautotrophic organism, total biomass or biomass product produced, cost of biomass or biomass product produced, or any combination thereof. The parameter that is optimized can depend on the economics of biomass production and utilization. Optimal conditions can be determined by monitoring growth conditions in a bioreactor and controlling the bioreactor.

Growth conditions that can be monitored can be selected from the group consisting of pH, light conditions, gas conditions, temperature, pressure, volume, biomass, and biomass products concentrations. The growth conditions can be monitored using any methods known by those skilled in the arts. In some embodiments of the invention, the growth conditions are monitored in real-time. The growth conditions can be monitored without user-intervention.

A bioreactor parameter can be controlled to optimize a growth parameter, or a bioreactor parameter can be controlled to maintain or achieve a growth condition. Optimization of growth of a photoautotrophic organism growing in a bioreactor can include identifying one or more growth parameters, determining an algorithm for evaluating the one or more growth parameters, monitoring one or more growth conditions in the bioreactor, determining if the one or more growth parameters can be improved, and altering a bioreactor parameter to optimize the one or more growth parameters. A growth parameter can include any growth parameter described herein, or for example, rate of biomass or biomass product production relative to time or relative to energy supplied to a light source, cost of biomass or biomass product production, or an amount of biomass or biomass product produced.

Bioreactor parameters that can be controlled can be selected from the group consisting of gas supply, heating or cooling, pressure, light supply, or nutrient supply. Control of a bioreactor parameter can affect one or more growth parameters described herein.

An increase in gas supply can increase the absorption of gas into a growth media for supporting growth of a photoautotrophic organism. For example, increased supply of carbon dioxide can increase the amount of carbon dioxide available to a photoautotrophic organism. Increased availability of carbon dioxide can increase the rate of carbon fixation by the photoautotrophic organism.

Amount of gas supplied to a bioreactor can be optimized based on any growth parameter described herein. For example, an increase in gas supply to a bioreactor resulting in an increased growth rate for a photoautotrophic organism growing in the bioreactor can suggest that the gas supply can be further increased to further increase the growth rate. Conversely, an increase in gas supply resulting in decreased growth rate can suggest that the gas supply can be decreased to increase the growth rate.

In some embodiments, altering carbon dioxide supply, in the form of solid carbonate, aqueous carbonic acid, or gaseous carbon dioxide can affect the pH of a growth media. By choosing between these forms of carbon dioxide, pH conditions of the bioreactor can be controlled.

Temperature can affect growth of a photoautotrophic organism. In some embodiments of the invention, temperature can be increased or decreased to change the growth rate of a photoautotrophic organism or any other growth condition described herein. In certain situations, increasing temperature can increase or decrease the rate of growth for a photoautotrophic organism. In other situations, change temperature can change the profile of biomass products produced. For example, reduction of temperature may lead to formation of fatty acids with a higher or lower degree of polyunsaturation.

A light source can be controlled for efficient growth of a photoautotrophic organism. Control of a light source can include controlling one or more wavelengths emitted by the light source or delivered to the bioreactor, controlling intensity of the light emitted by the light source or delivered to the bioreactor, and controlling intermittent emission of light or delivery of light to the bioreactor.

A bioreactor of the invention including a container used for growth of a photoautotrophic organism can be operated by adjusting one or more wavelengths of light reaching the container to provide at least one or more wavelengths of light that support growth of the photoautotrophic organism. The one or more wavelengths of light reaching the container can correspond to one or more peak absorption wavelengths of at least one light absorption pigment of the photoautotrophic organism.

The one or more wavelengths can be optimized or chosen based on a growth parameter described herein. The one or more wavelengths can be tuned for the production or rate of production of biomass or one or more biomass products. In some embodiments of the invention, a first set of one or more wavelengths can be delivered for production of biomass and a second set of one or more wavelengths can be delivered for production of a biomass product. For example, production of biomass can utilize a set of wavelengths between 400-500 nm and production of a biomass product can be between 500-700 nm.

The amount of the light emitted by the light source or delivered to the bioreactor can be controlled. The amount of light can be between 50 μEm⁻²s⁻¹ to 10,000 μEm⁻²s⁻¹, 100 μEm⁻²s⁻¹ to 7,500 μEm⁻²s⁻¹, or 150 μEm⁻²s⁻¹ to 5,000 μEm⁻²s⁻¹. The amount of the light delivered can be adjusted such that the amount of light delivered to a photoautotrophic organism is an amount of light that can be utilized by the photoautotrophic organism.

The intermittent emission of light or delivery of light to the bioreactor can be controlled for optimal growth of the photoautotrophic organism. The intermittent emission of light can be regular or irregular. For regular intermittent emission of light, the frequency can be from 0.05 to 2000 Hz, 5 to 1000 Hz, or 10 to 500 Hz. Light can be emitted 1-95%, 5-90%, or 10-80% of the total time. The intensity of the light over time can be described by a light intensity waveform. The light intensity waveform can be triangular, saw-tooth, square, sinusoidal, or any other desired shape.

The supply of light can also be controlled by changing the arrangement of one or more light conducting channels used to transmit and distribute light within the bioreactor. The light conducting channels can be spaced closer or further apart or in a uniform or non-uniform pattern. The arrangement of the light conducting channels can allow for increased concentration of photosynthetic organism to be grown by improving the distribution of light in cultures of photoautotrophic organisms. For example, arranging the light conducting channels in a bioreactor such that the light conduction channels are closer together can increase the concentration of photosynthetic organisms that can be grown in the bioreactor.

A growth profile of a photoautotrophic organism introduced to a bioreactor with one or more light sources can be monitored over time and the light source can be adjusted over time. The growth profile can include of measurements of photoautotrophic organism concentration in the bioreactor over time. The one or more light sources can be adjusted based on the growth profile of the photoautotrophic organism. Adjustments include changing the one or more wavelengths of light, changing the intensity of light emitted, changing the pattern of intermittent light emission, and/or changing the arrangement of one or more light sources. The adjustments can be performed to maximize growth rate.

In another example, an amount of biomass product produced by a photoautotrophic organism introduced to a bioreactor with one or more light sources can be monitored over time and the light source can be adjusted over time. Biomass products can be any biomass product described herein. The one or more light sources can be adjusted based on the growth profile of the photoautotrophic organism. Adjustments include changing the one or more wavelengths of light, changing the intensity of light emitted, changing the pattern of intermittent light emission, and/or changing the arrangement of one or more light sources. The adjustments can be performed to maximize biomass product or biomass production rate.

The control of a bioreactor parameter can be performed using a computer or other electronic means described herein. Changing a setting for a bioreactor parameter on a computer can cause an electronic signal to be delivered to a device on the bioreactor that can alter the bioreactor parameter. For example, submitting an increase in gas supply to 5 volumes of gas per volume of bioreactor on a computer can cause a valve controlling an actual rate of gas supply to the bioreactor to increase the rate of gas supply to 5 volumes of gas per volume of bioreactor.

In some embodiments of the invention, the optimization of growth conditions for growing a photoautotrophic organism allows for specific amounts of biomass and biomass product to be produced relative to a growth time, volume of growth media used for growth of the photoautotrophic organism, and/or energy supplied to the bioreactor or bioreactor light sources. The optimization of growth conditions includes optimizing light delivery, nutrient delivery, and other growth conditions described herein.

Using the bioreactors of the invention with optimized growth conditions described herein, a biomass production efficiency of no less than 100, 10, 1, 0.1, or 0.01 milligrams of biomass can be produced per Id of light energy delivered within the bioreactor.

Using the bioreactors of the invention with optimized growth conditions described herein, a biomass production efficiency of no less than 50, 5, 0.5, 0.05, or 0.005 milligrams of biomass can be produced per kJ of energy supplied to the light source. The photoautotrophic organism used to produce the biomass can be genetically modified such that photon absorption capability is enhanced as compared to a corresponding wildtype photoautotrophic organism. The photoautotrophic organism can be genetically modified to have enhanced biomass production capability as compared to a corresponding wildtype photoautotrophic organism. The production capability can be evaluated based on time, total production, or on energy supplied to the light source. Genetic modifications can allow for a narrow band of light, such as 5, 10, or 15 nm or less, to be used to grow a photoautotrophic organism.

In other embodiments of the invention, the photoautotrophic organism can be grown to a concentration of more than about 0.3, 1, 3, 5, 10, 15, 30, 50, 75, 125, 175 or 200 grams of biomass per liter of medium. This concentration of biomass can be grown in less than 50, 40, 30, 20, 15, or 10 hours. The concentration of biomass per liter of medium that can be grown can be determined by using the Beer-Lambert law, a molar absorptivity for the photoautotrophic organism, and an intensity of light required for growth. Additional assumptions can be made such as that a layer of non-growing photoautotrophic organism can form near a light source and that light transmitted through the layer of non-growing photoautotrophic organism needs to be of an intensity that is required for growth.

### Methods for Biomass Collection

The invention includes methods for harvesting biomass from a bioreactor comprising using a biomass collector. The biomass can be a photoautotrophic organism. The biomass collector can be fixed to a frame or fixed to the bioreactor. In some embodiments of the invention, the biomass collector is fixed to a container of the bioreactor. The method can include utilizing the biomass collector for separating the photoautotrophic organism from a growth media used to grow the photoautotrophic organism. The biomass collector can include a movable unit that preferentially captures the photoautotrophic organism. The movable unit can be activated, allowing for the movable unit to translate along a length of the container such that biomass, in the form of a photoautotrophic organism, is concentrated on one side of the movable unit to form a solution of concentrated photoautotrophic organism. The movable unit can be constructed of a perforated material or a mesh material for the selective capture of the photoautotrophic organism. The bioreactor can also include a harvest port for harvesting the solution of concentrated photoautotrophic organism from the bioreactor. The harvest port can extend from a length of the container. The harvesting of biomass can be performed in less than 3, 2, 1, 0.5, 0.25, or 0.1 hours.

Activating the movable unit can be performed remotely from a computer or performed using controls on the biomass collector. By translation of the movable unit, a photoautotrophic organism can be concentrated on one side of the movable unit. The movable unit can translate a distance less than 98, 95, 90, 80, 70, 60, 50, 40, or 30% of a length of the bioreactor. Movement of the movable unit can be driven by a worm drive or a cable. The worm drive or the cable can be implemented using any methods known to those skilled in the arts.

The movable unit can be configured such that the photoautotrophic organism can be concentrated about 1-15, 1.2 to 10, or 1.5 to 5 times the original concentration prior to translation of the movable unit to form the solution of concentrated photoautotrophic organism. The photoautotrophic organism can be concentrated to a degree such that the photoautotrophic organism that was suspended in a growth media is still suspended in the growth media and can be harvested by pumping the solution of concentrated photoautotrophic organism out of the bioreactor.

The solution of concentrated photoautotrophic organism can be optionally loaded into a holding tank. The holding tank can be used for separating the photoautotrophic organism from the growth media by gravity to form a solution of further concentrated photoautotrophic organism.

In some embodiments of the invention, the solution of further concentrated photoautotrophic organism or the solution of concentrated photoautotrophic organism can be processed for recovery of a biomass product. The biomass product can be any biomass product described herein. The biomass product can be a liquid biomass product, such as oil or straight vegetable oil, and the biomass can be algae.

A hydraulic ram press can be used for recovery of a biomass product. The hydraulic ram press can comprise a pressure driven plate that compresses biomass against a permeable material. The permeable material can comprise multiple pores that allow for growth media and other liquid components to escape while retaining solid biomass. The pores can be of a size that allows for more than 95%, 90%, 85%, 80% or 75% of the biomass to be retained while more than 60%, 70%, 80%, or 90% of the growth media and liquid components are removed. The growth media and liquid biomass product can be collected in a holding tank. If the liquid biomass product is immiscible with the growth media, then the liquid biomass product can be easily separated from the growth media by pouring off or aspirating the growth media or the liquid biomass product. Growth media, including water, can be returned to the bioreactor. Growth media separated in the holding tank can be returned to the bioreactor.

In some embodiments of the invention, gases exiting the bioreactor can include a biomass product. Unused gas supplied to the bioreactor or gases produced by the photoautotrophic organism can exit the bioreactor through one or more exit ports. A pressure relive valve or any other control valve can control pressure inside the bioreactor and a rate of gas exit from the bioreactor. The gas exiting the reactor can be compressed or used immediately.

### Methods for Using Biomass

A variety of biomass products can be extracted from biomass derived from a photoautotrophic organism grown in a bioreactor described herein. For example, a biomass product can be selected from the group consisting of a press cake, an oil, a vegetable oil, an omega 3-fatty acid, a triacylglycerol, a docosahexaenoic acid, an amino acid, a small molecule, an antioxidant, an organic dye, an isoprenoid, a carotenoid, a vitamin, a hormone, a carbohydrate, a protein, a gas. These biomass products can be utilized in numerous applications. The following embodiments are included by way of example only and are not intended to be limiting in scope.

Press cake can be utilized for as a source of combustible biomass material. Press cake can be combusted to form carbon dioxide and water, and the gases formed can be used in a bioreactor for growth of a photoautotrophic organism.

Oil, such as vegetable oil, can be used as a biodiesel, a combustible biomass material, or in cooking applications. The vegetable oil can be processed using any methods known to those skilled in the arts or used without any further processing.

An amino acid can be utilized in nutraceutical applications or in bioremediation applications. Amino acids produced can include alanine, cysteine, aspartic acid, glutamic acid, phenylalanine, glycine, histidine, serine, valine, and tyrosine. The amino acid can be hydrophilic or hydrophobic.

An amino acid or a small molecule can be utilized as a chelating agent. A chelating agent can be used for bioremediation of suspended metals, heavy metals, and/or radioactive isotopes. An amino acid chelating agent can be ethylenediaminetetraacetic acid (EDTA). A small molecule chelating agent can include alpha-lipoic acid (ALA) and/or aminophenoxy ethane-tetra acetic acid (BAPTA).

A biomass product, like a vitamin or a hormone can be utilized in nutraceutical applications. A vitamin can include ascorbic acid and vitamin E. Vitamin E can also be called tocopherols and/or tocutrienols. A hormone can include melatonin.

An organic dye can include chlorophyll, carotene, and quercetin. Organic dyes can be used as a pigment for coloring materials as labeling reagents.

A carbohydrate extracted from biomass can include glucose, cellulose, or starch. The carbohydrate can be used as a source of energy for growth of other organisms, as a nutraceutical, in cooking applications, or any other type of application that utilizes carbohydrates.

A protein that can be extracted from biomass can include ribulose bisphosphate carboxylase-oxygenase (RuBisCO) and/or acetyl-Coa carboxylase (ACCase). ACCase participates in conversion of malonyl-CoA to acetyl-CoA carboxylase and can be helpful in increasing fatty acid production.

Gas exiting the bioreactor, for example unused carbon dioxide, oxygen produced by the photoautotrophic organism, or any other gas, can be collected and used in other processes. As an option, oxygen evolved from photoautotrophic organisms grown in the bioreactor can be captured and used in environments that have limited amounts of oxygen such as closed terrestrial environments, extra-terrestrial environments, or aqueous environments. Collecting oxygen evolved from the photoautotrophic organisms grown within the bioreactor and using the gas can reduce the chance of introducing contaminants from other environmental sources.

Methods of the invention also include utilizing combusted biomass for growth of organisms as nutrients or fertilizer. For example, flue gases such as carbon dioxide, nitrogen oxides, and sulfur oxides can be delivered to a bioreactor for growth of a photoautotrophic organism. Alternatively, combusted biomass in solid form can be used as a fertilizer for growth of plants or other organisms.

The methods of the invention provide for producing energy using a manufacturing plant comprising growing a photoautotrophic organism in a bioreactor for producing a biomass or biomass product, using a power plant to produce electricity from the biomass or biomass product, and supplying electricity and carbon dioxide to the bioreactor for production of biomass or biomass product.

In other embodiments of the invention, methane gas produced by digesting or fermenting biomass can be combusted to produce carbon dioxide. The carbon dioxide can be supplied to a bioreactor of the invention for growth of a photoautotrophic organism. The digested or fermented biomass can be used as agricultural fertilizer.

### EXAMPLE 1

### A Bioreactor Powered by Photovoltaic Panels

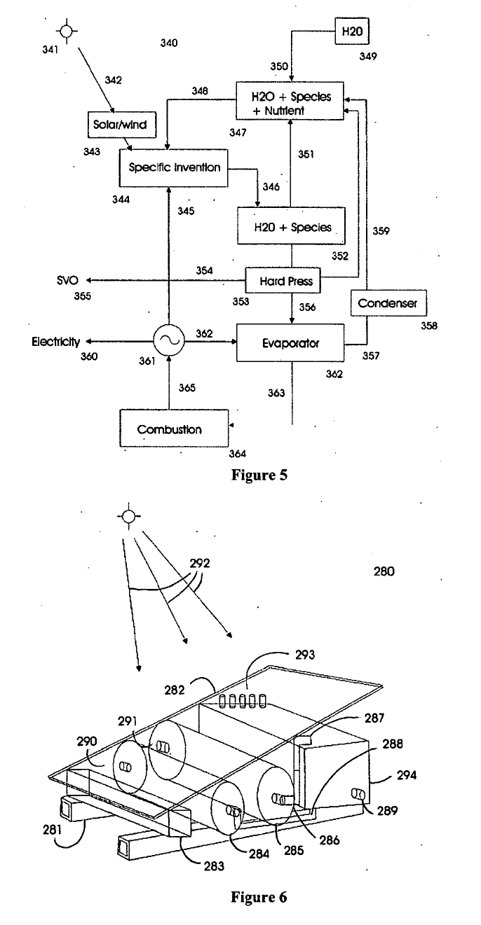

FIG. 6 shows a bioreactor system deployed on a skid (281). The bioreactor system can comprise a bioreactor (294) and a solar photovoltaic panel array (282) that produces electricity during sunlight hours that is used directly, and charges a battery bank with controller (283). The electricity can be used to power the bioreactor system and all components therein. The bioreactor can comprise a light emitting source for supplying light to photoautotrophic organisms growing in one or more containers of the bioreactor. The one or more containers, a light emitting source, all electrical interconnection, and a computer loaded with the computer control programs to control the bioreactor system can be housed in an enclosure. Oxygen vents (293) can be used to vent unabsorbed gas and oxygen from the bioreactor.

A media tank (286) containing a growth media and the species of autotroph to be cultivated can be mounted on the skid. An intake valve (291) and output pipe and pump (287) can be used to transfer materials between the media tank and the one or more containers. Feedstock gas or gases can be contained in a pressure vessel (284) that has an output valve and conduit (285) leading to the bioreactor (294) and an input valve (290) to receive gas or gases from an external source.

### EXAMPLE 2

### Energy Cycle for Using Solar Energy to Produce Energy and Biomass Products

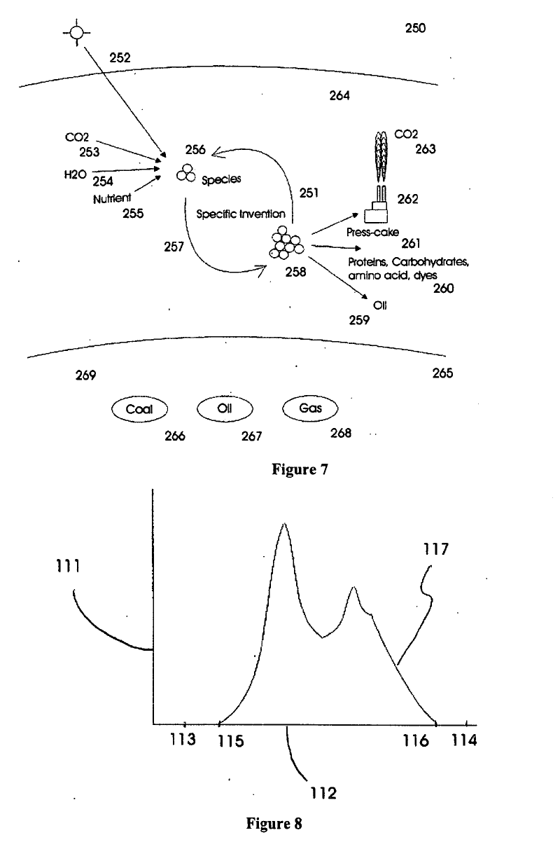

FIG. 7 shows an energy cycle for producing renewable energy. Nature chose to sequester fossil fuels in the earth (265). Coal (266), oil (267), and natural gas (268) are used world wide for chemical fuels and increases the carbon dioxide (253) and other greenhouse gas concentrations in the atmosphere (264). Exo-atmospheric carbon, such as coal, oil, and natural gas, can be replaced with a renewable energy sources, such as biomass (258). Biomass (258) can be produced using a process (257) that utilizes an optimized photosynthetic process in a bioreactor. The photosynthetic process can utilize nutrients (255), water (254), and light (252). Photoautotrophic organisms can be grown using the optimized photosynthetic process (258) and can be processed for oil (259), protein, carbohydrate, amino acids, dyes, and other organic compounds (260), and press cake (261). The press cake can be used for animal feedstock, or burned in coal-fired power plants as an alternative fuel. This press cake fuel (261) can be combusted and used as gas feedstock for the bioreactor. The photoautotrophic organism can be algae, grown to produce straight vegetable oil and press cake.

The sun can produce the light (252) that is used directly or indirectly for photosynthesis. Light from the sun can be converted to electricity through solar photovoltaic panels and then used to power artificial light sources. This electricity is used directly or indirectly. Under some instances, electricity produced by photovoltaic panels is used to charge battery banks that provide power on demand to run valves, pumps, sensors, displays and the artificial light sources that photosynthesis. The present invention is a global solution providing pollution mitigation and carbon neutral biofuels.

### EXAMPLE 3

### Spectrum of Light Emitted by a White Light Emitting Diode

FIG. 8 shows a spectral distribution for a white light emitting diode. The vertical axis (111) is number of photons or relative luminescence intensity. The horizontal axis (112) represents wavelength. Position 113 represents 300 nm, which is the lower limit for photosynthetically available radiation. Position 114 represents about 800 nm. The white light emitting diode spectrum (117) falls between positions 115 and 116, which are between 113 and 114.

The sun has a temperature of approximately 5,774 Kelvin. The white light emitting diode has a distributed spectrum that approximates a 6,000 degree Kelvin blackbody radiator. The white light emitting diode can match the natural blackbody radiance of the sun.

### EXAMPLE 4

### Example of a Bioreactor

FIG. 18 shows a bioreactor (31) with a means of controlling and optimizing the cultivation of a given photoautotrophic organism for maximum growth in a minimum amount of time. The growth cycle can be controlled by a computer (63) and computer program described herein (64) that interfaces with the bioreactor through a serial port (61) and a suitable cable (60) that connects to the bioreactor (31) through a suitable port, such as an RS232 connector (59). The bioreactor is configured to allow for control of light provided for driving photosynthesis inside the bioreactor (31).

Aqueous media can enter the bioreactor (31) through pathway column (33). Media composed of water, nutrients and a photoautotrophic organism can be premixed. An input valve (34) controls the input of the water, nutrients, and photoautotrophic organism. An output valve (37) provides regulation of exit media through exit column (36). The input valve (34) and output valve (37) are controlled by the computer and computer program. Power to all components is produced from solar photovoltaic panels.

Light emitting diode arrays (40) and (39) irradiate the bioreactor (31) through transparent side walls (41) and (32), respectively. The light emitting diode arrays can comprise one or more light emitting diodes (42, 43, 44, 45, 46, 47). The light emitting diodes emit photons of one or more wavelengths chosen for their range between 300 and 700 nm of wavelength. Groups of light emitting diodes of similar material are wired within the arrays (39) and (40), providing individual string arrays that are energized at command of the computer software (64) that controls the production cycle of photoautotrophic organism within the bioreactor.

A sensor element (56) is a top float switch that when activated, indicates the bioreactor (31) vessel is filled with media.

A CO₂ input valve (50), also controlled by the computer program, regulates input gas to be diffused into the media contained within the bioreactor (31). A gas sparger (48) extends a length of the bottom of the bioreactor (31) and is perforated with many small holes along a length and width of the sparger. These small holes produce small bubbles when pressurized with an input gas.

The input gas can be air, flue gases, CO₂ and other sources of CO₂ gas for consumption by the photoautotrophic organism. The micro-bubbles or small bubbles produced by the perforations in the gas sparger (48) produce cones of micro-turbulence that mix the photoautotrophic organism and improve exposure of the photoautotrophic organism to light emitted by the light emitting diode arrays (39) and (40), thus stimulating photosynthesis.

The sensor elements can also be temperature and pH sensors that are connected to the computer (63) through electrical connections. A sensor (58) is a lower float switch. When the sensor (58) is switched off, then the computer program is signaled and an exit port (37) is closed. In this configuration a portion of the media in the bioreactor at the end of the production cycle is left in the bioreactor (31) to seed a next production cycle.

The bioreactor herein disclosed (31), can be constructed using any means known to those skilled in the arts and can be controlled using a computer or any other means known to those skilled in the arts. The bioreactor provides increased utility over the state-of-the-art by controlling the dominate factors of a photoautotrophic organism's life cycle. These factors include media, light delivery, temperature, pH, and gas supply. All of these factors are controlled to maximize production of the photoautotrophic organism by optimization of photosynthetic activity.

The software can be committed to removable software media (62) and can be customized for growth of one or more photoautotrophic organisms. The bioreactor is a universal platform that is tunable to optimize production for any photoautotrophic organism. The bioreactor offers novel and significant functions for the practical production of photoautotrophic organism biomass at industrial scales.

### EXAMPLE 5

### A Bioreactor with Multiple Containers and Multiple External Light Sources

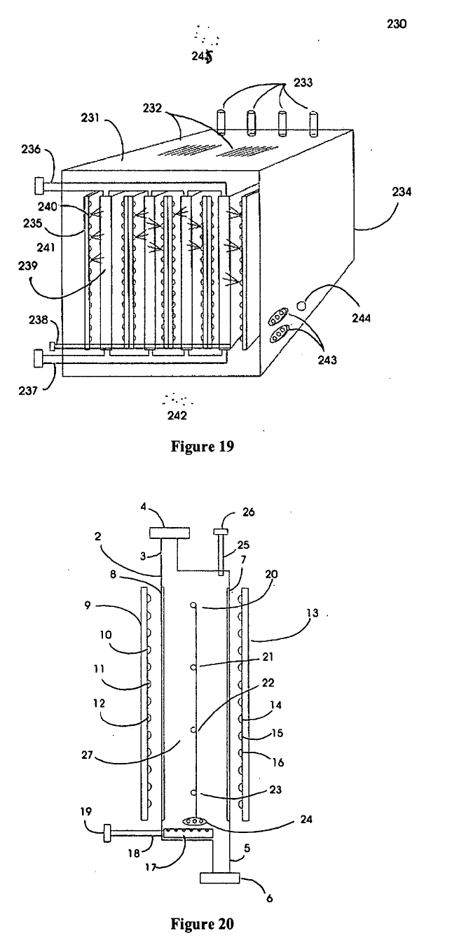

FIG. 19 shows another embodiment of a bioreactor with an array of bioreactor vessels. The array of bioreactor vessels comprises multiple containers (239) and multiple external light sources (235). An enclosure (231) can house the multiple containers and external light sources and can control thermal conditions of the containers holding a growth media for growth of a photoautotrophic organism. Gas can be supplied to the containers through a gas intake port (238) and filled with growth media through a water and nutrient intake port (236). Growth media and photoautotrophic organisms grown in the bioreactors can be harvested through an exit port (237).

As described herein, sensors, and control valves are interconnected to RS232 serial ports (243). Power for the light emitting diodes, produced using solar energy, is supplied through a power plug (244). The power can be regulated and controlled by a computer program.

The equilibrium temperature of the containers (239) can be regulated by opening and closing vents (232) positioned at the top, and bottom of the enclosure (231). Active means of temperature control by introducing cooler or warmer air, known to those skilled in the arts, can be adopted as a common means of temperature control. Means of temperature control can incorporate heat produced by the external light sources (235).

### EXAMPLE 6

### A Bioreactor with an Array of Light Sources

FIG. 20 shows an illustration of a bioreactor comprising an array of lights and a container for growing a photoautotrophic organism. The container (2) comprises any material known by those skilled in the art, such as high density polyethylene, plastics, or glass. The materials are suitably strong and chemically neutral to the cultivation of photoautotrophic organisms. The bioreactor comprises an input column (3) for delivery of liquids and other nutrients necessary for introducing a growth media to the bioreactor and an output column (5) for removal of the photoautotrophic organism and growth media. An input column valve (4) and an output column (6) control the flow of liquid through the input and output columns. The valves are controlled by a computer via electrical connections between the valves and the computer. The bioreactor further comprises optically transparent side elements (7, 8) that are composed of glass, Plexiglass, or other suitable materials known in the art to transmit light at least in the range of 300 nm to 700 nm. This range of photon transmission allows one or more wavelengths to be transmitted by one or more light sources (10, 11, 12, 14, 15, 16) on an array of light sources (9, 13) to the bioreactor. The one or more wavelengths are chosen to maximize the growth of the photoautotrophic organism.

The arrays of light sources provide controllable wavelength spectra to be irradiated on the chosen photoautotrophic organism to be cultivated. The one or more light sources are the same as each other or different. The one or more light sources are controlled by a computer and can emit a range of wavelengths and intensities of light.

Gas supply to the bioreactor is supplied to an input port (19) and fed through a connecting pipe or tube (18), and then enters the bioreactor through a gas sparger (17). The gas sparger comprises multiple small holes that have a diameter less than 0.5 cm that allows for distribution of gas to the growth media in micro-bubbles. The small holes, when pressurized, produce micro-bubbles that rise through the bioreactor (2) chamber and diffuse the input gas or gases into the media (27) contain therein. The micro-bubbles produce additional utility in that cones of turbulence and micro-turbulence are produced in the media (27) effectively mixing the media and increasing exposure of a photoautotrophic organism growing inside the bioreactor to one or more wavelengths of light.

The bioreactor includes an outgas column (25) with optional sensors and an output gas port (26). Gases exiting the bioreactor include oxygen and unabsorbed source gas or gases from input port (19). These gases are vented or diverted for other purposes, such as oxygen extraction.

The bioreactor comprises an upper float switch (20) and lower float switch (23) for determining volume of liquid inside the bioreactor, an electrical connector (24) for communicating with the electrical hardware of the bioreactor, such as an RS232 connector or an ethernet connector, a sensor array (21) for measuring temperature, pH, light conditions, and pressure. The sensor array can be mounted to a wall of the bioreactor. Upper float switch (20) can be used for determining a high volume and lower float switch (23) can be used for determining a low volume.

### EXAMPLE 7

### An Example of a Bioreactor Configure for Use with an External Light Source

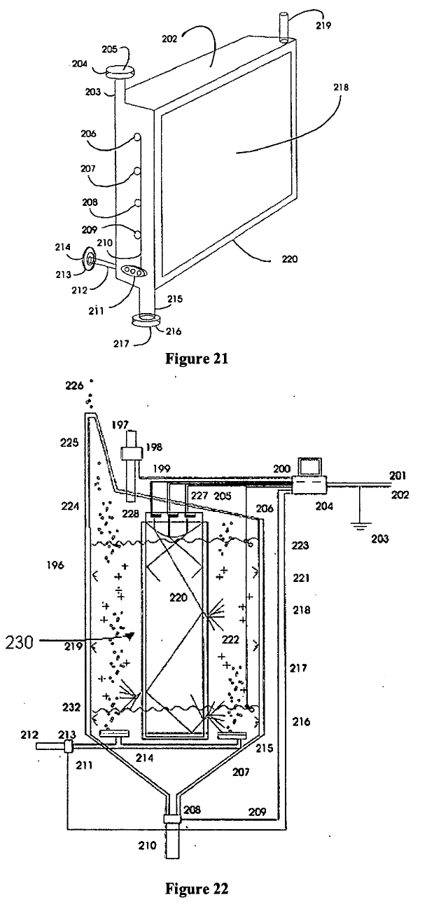

FIG. 21 shows a bioreactor (202) fabricated with materials known in the art, such as high density polyethylene, or other materials such as other plastics. With a width exceeding a depth of the bioreactor, the bioreactor is molded with internal slots, or other known means for guiding or fastening transparent lateral walls (218). The opposite transparent lateral wall is not shown.

The transparent lateral walls (218) can be made of glass or Plexi-glass, or other known materials. The bioreactor can be fitted with an intake column (203) and connected to a controllable valve (204) to regulate flow of an aqueous media, containing a growth media and an initial concentration of photoautotrophic organisms. The lower region of the bioreactor (202) is fitted with an exit column (215) providing a pathway for media to exit through. The exit of media is regulated by a controllable valve (216) connected to the exit column (215).

Exiting media will have a higher concentration of photoautotrophic organisms per unit volume of aqueous solution as compared to the initial concentration of photoautotrophic organisms. FIG. 21 shows a gas intake conduit (212) connected to a gas intake port (214) for supply of a gas to the bioreactor. A controllable valve (213) can be connected to the gas intake conduit (212) to regulate the flow of gas to a gas sparger (211).

The bioreactor (202) is fitted at the upper end with an exit gas vent (219). This vent (219) can be passive or active. The vent can be used for release of excess oxygen, which is a by-product of photosynthesis, and gas or that is not used by the photoautotrophic organism. Vent (219) also acts as a pressure relief valve allowing internal pressure to be controlled.

The bioreactor can be controlled by a computer and a computer control program described herein. Components of the bioreactor, including an upper float switch (206), a temperature probe (207), a pH sensor, a photosensor (208), a lower float switch, and valve controls (204, 213, 216) are attached to an appropriate serial port RS232 connector (211) for monitoring and control of bioreactor growth conditions by the computer.

The bioreactor (202) can be a status feedback instrument providing status on all desired metrics, such as temperature, pH, and biomass production as a function of time. This can allow for the selection of desired wavelengths produced by light emitting diode arrays. The bioreactor can be powered by solar energy converted to electricity. The electricity can be stored in rechargeable batteries for supply of power on demand.

The bioreactor (202) can be fabricated separately from the light emitting diode arrays. This can increase safety in assembling the bioreactor. Racking guideways (220) for supporting light emitting diode arrays can be mounted on the bioreactor (202). The light emitting diode array can slide in and out of the racking guideways.

### EXAMPLE 8

### A Bioreactor with a Light Conducting Channel Controlled by a Computer

FIG. 22 shows a cross-sectional view of a bioreactor with an electro-optical apparatus for the production, control, and distribution of light of known wavelength, duration, and intensity to maximize growth of a photoautotrophic organism.

The bioreactor can be controlled by a computer system. The computer system includes software and hardware with connectivity (200) to valves (198, 213, 208), sensors (216, 223), and light sources (228) of the bioreactor. Computer hardware and software (204) can also monitor and control other growth conditions of the bioreactor. The system can be powered by electrical energy through positive and negative leads (201, 202). One of the leads can be connected to a ground (203).

Light produced by a light emitting diode array (228) can be directed toward the bottom of the light conducting channel (230). Light (220) that travels through a light conducting channel (230) and reaches the edge of the light conducting channel is absorbed, reflected, or transmitted. Light can be transmitted to the interior of the bioreactor (222), thus stimulating growth of the photoautotrophic organism. The bioreactor has internally reflecting surfaces (219) for containing light within the bioreactor.

The computer system (204) controls all active elements of the bioreactor, such as valves (198, 208, 213) and real time sensors, such as float switches (223, 216).

An input valve (198) is opened to fill the bioreactor with a growth media and a photoautotrophic organism. When liquid level is above an upper float switch positioned at an upper liquid level (223), the upper float switch is switched on and the input valve (198) is closed. Closing the input valve (198) triggers the software to activate the light emitting diode array (228) to generate light (220). Light can be delivered using any methods described herein.

Flue gas, CO2, NOX, SOX, and other gases are introduced to the bioreactor under pressure to a gas input port (212), controlled by a gas valve (213), and dispersed within the growth media through a gas sparger (214). Through photosynthesis, the photoautotrophic organisms growing in the bioreactor produce oxygen. Excess oxygen and other unabsorbed gases are released through an exit gas port (197).

After sufficient time, the light emitting diode array (228) is turned off and the photoautotrophic organisms are exposed to total darkness. This step can be omitted if light was delivered to the bioreactor in intermittent pulses. At the end of growth, an output valve (208) opens for harvest of the photoautotrophic organism and emptying of the bioreactor using gravity. As liquid level drops below a lower liquid level (232), the lower float switch is switched off and causes the output valve (208) to close.

### EXAMPLE 9

### A Bioreactor with a Container Having Reflective Walls and an Internal Light Conducting Channel

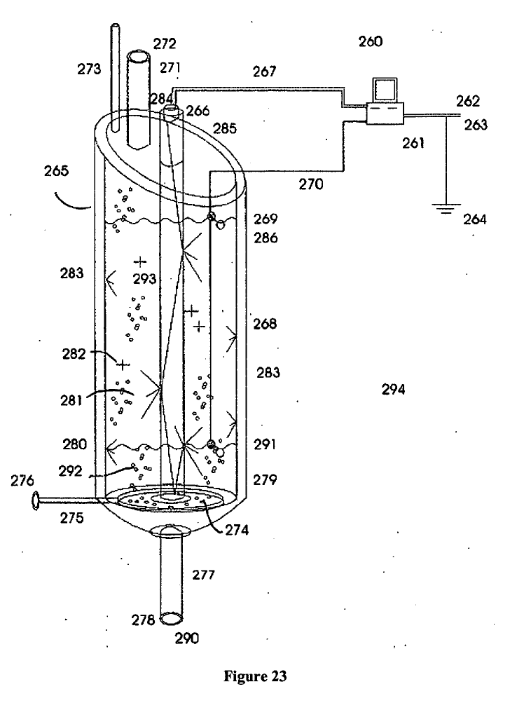

FIG. 23 shows another embodiment of a bioreactor (265). A computer system (261) is engaged to control renewable electricity delivered as a voltage across positive lead (262) and negative lead (263) connected to ground (264) is used to power in precise and controlled sequence electrical loads of the present invention for mass-transfer volume management for liquids, gases, solids, and radiation for autotroph cultivation.

As shown in FIG. 23, the bioreactor vessel (265) comprises a light conducting channel and light source (266) that is controlled and powered by leads (267) originating from the computer system (261). The light source deliver light to the interior of the bioreactor vessel (265) to stimulate growth of a photosynthetic organism, including diatoms, autotrophs, photoautotrophs, chemoautotrophs, heterotrophs or any photosynthetic species or groups of species. Upper float switch (269) and lower level float switch (291) indicate to the computer system via transmission and power wires (270) their status and therefore fluid levels in the bioreactor (265).

The bioreactor vessel (265) has a water and nutrient intake valve (271) allowing water, nutrients, and photoautotrophic organisms to enter the bioreactor vessel (265). Output pipe (277) terminates with an output valve (278), which is also computer controlled. Flue gas, CO₂, Air, or other gas or gases are pressurized and enter the vessel (265) though intake gas valve (276). Opening gas valve (276) pressurizes a sparger (274) and gas (292) bubble up through the bioreactor (265) to interact with the photoautotrophic organism growing in the bioreactor (293).

The interior surfaces of the bioreactor (265) are coated with a reflective material (268) causing photons that impinge on the inner wall of the bioreactor (265) to reflect back into the bioreactor.

The bioreactors described herein can be arranged in a subarray of bioreactors as shown in FIG. 33. The bioreactors can be connected such that the inputs and outputs of the bioreactors are fluidly connected. Growth in subarrays of bioreactors can allow for efficient growth of photoautotrophic organism.

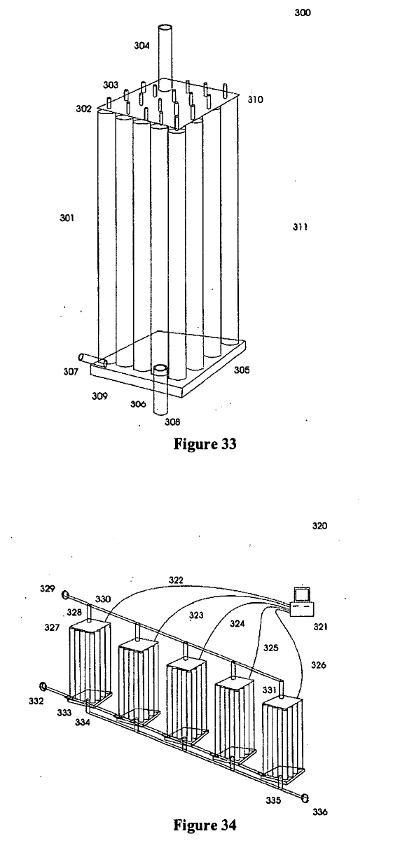

Subarrays of bioreactors can be arranged to form an array of bioreactors, as shown in FIG. 34. Similar to the arrangement of bioreactors within a subarray, inputs and outputs of subarrays can be fluidly connected.

### EXAMPLE 10

### Example of an Assembly of Light Conducting Channels and Gas Sparging Devices

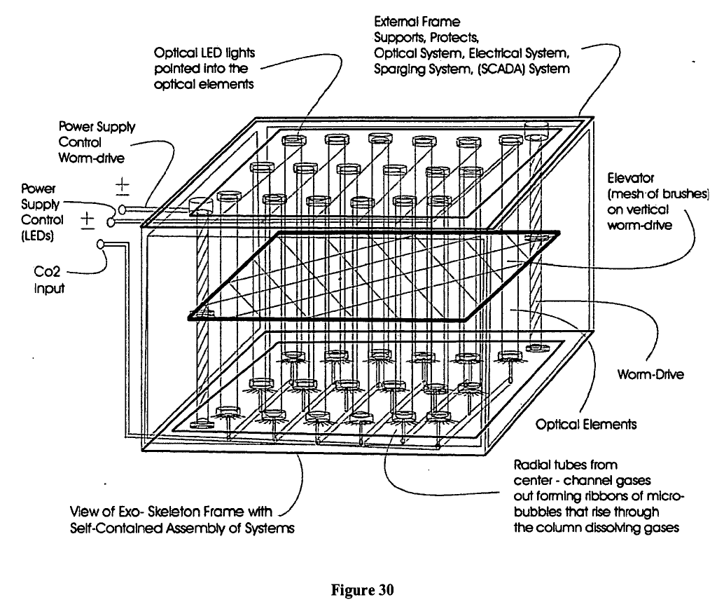

FIG. 30 shows an example of an assembly of light conducting channels that can be packaged and used with any type of container or immersed in any body of water for growth of a photoautotrophic organism, such as a pond, a lake, or a reservoir. The assembly can comprise an external frame that can be used to support an optical system, an electrical system, a control system, a gas sparging system, and a biomass collector. The optical system can comprise one or more light conducting channels and one or more light sources. The one or more light sources can be LED lights positioned to direct light to the one or more light conducting channels. The electrical system can comprise a power supply and electrical wiring for powering the bioreactor. The control system can be a SCADA system described herein, or any other control system for monitoring and controlling bioreactor growth conditions. The control system can also comprise any monitoring and controlling devices described herein. The gas sparging system can comprise gas input ports, such as a carbon dioxide supply port, and one or more radial tubes that are placed at the base of a light conducting channel. Gas can be sparged from the radial tubes and travel upward in a growth media near the exterior surfaces of the light conducting channels. The biomass collector can comprise an elevator or movable unit that can be used to concentrate a photoautotrophic organism grown in a bioreactor utilizing the assembly of light conducting channels and to clean the light conducting channels. The elevator or movable unit can be driven by one or more worm drives supported by the external frame.

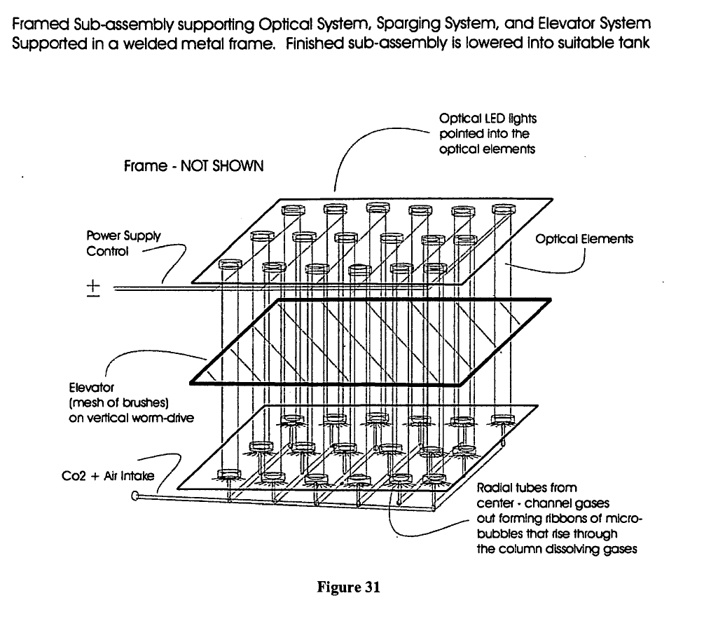

FIG. 31 shows the assembly depicted in FIG. 30 without the external frame.

### EXAMPLE 11

### Steps for Controlling a Bioreactor Process

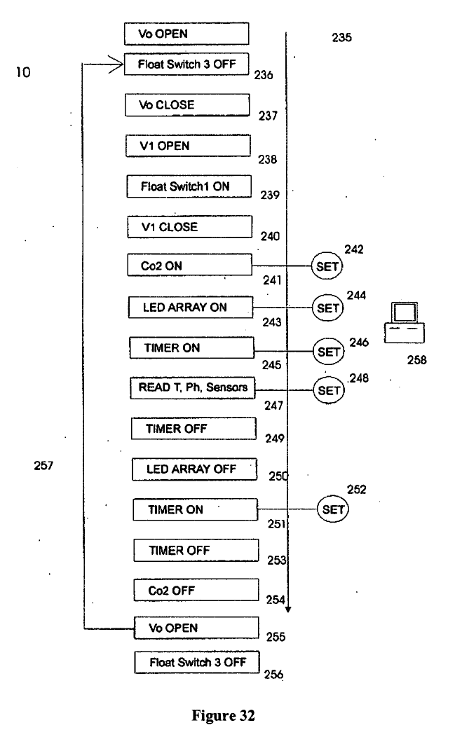

FIG. 32 shows a sequence of steps for controlling a bioreactor. The control can be performed by a computer (258) utilizing a software program that can be written in Fortran, C+++ or any other known programming language.

An output valve on a bioreactor is opened, designated by Vo open in FIG. 32, and growth media with or without a photoautotrophic organism is emptied from the bioreactor. Once the growth media is emptied, and liquid levels drop below the level of a lower float switch, the lower float switch is switched off (236) and triggers the output valve (237) to close. This action triggers an input valve to open (238) and stay open until water level in the reactor rises above an upper float switch and triggers the upper float switch to be switched on (239).

This triggers the input valve to close (240) and a gas intake valve to open (241). Gas, such as carbon dioxide, is bubbled through the growth media in the bioreactor. The gas intake valve is controlled by a computer setting (242). At the same time, a light emitting diode array or other light source is activated (243) and controlled by a computer software setting (244). The supply of light can be controlled as described herein.

A timer can be set (245) and monitored by the computer (246). Growth conditions are monitored (247) and communicated to the computer (248).

After a desired amount of time (246) or after desired growth conditions have been reached, the light emitting diode array is powered off (250). A timer for a dark period is set (251) and monitored by the computer (252). At this time, the gas input can be switched from carbon dioxide to air. After a period of time, a time limit can be reached (253) and supply of gas can be turned off (254). The output valve can be opened (255) for removal of growth media containing photoautotrophic organism. Once the lower float switch is switched off, the cycle can be repeated.

### EXAMPLE 12

### Growth Cycle for a Photoautotrophic Organism

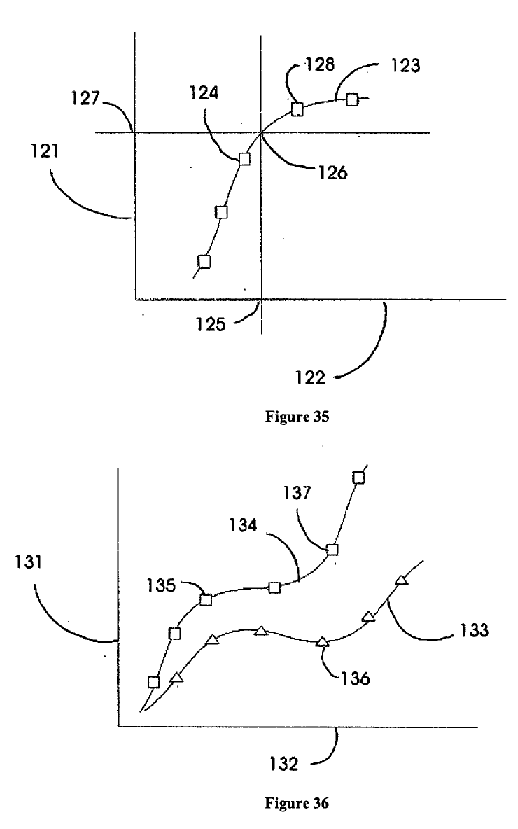

FIG. 35 is a plot of a typical growth profile of a given photoautotrophic organism. The plot shows a vertical axis of biomass concentration of photoautotrophic organism defined as grams per milliliter of water (131) with a horizontal axis of time (132) an autotroph growth rate.

Data points 134, 135, 136, 138, and 139 are taken over time and plotted, showing a growth profile. The photoautotrophic organism growth rate is maximum at point 135 and total growth is maximum at point 139. A knee in the growth profile occurs at point 137.

A point to which the photoautotrophic organism is grown depends on the cost basis of additional growth. In some cases, harvesting the biomass at point 135, 137, or 139 is optimal. In some cases, time for a production cycle is considered.

### EXAMPLE 13

### Two Growth Profiles for a Growing Photoautotrophic Organism

FIG. 36 shows a plot of two growth profiles corresponding to growth of a photoautotrophic organism at two different light intensity levels. The vertical axis (131) represents concentration of photoautotrophic organism in grams per mL of aqueous solution and the horizontal axis (132) is time. The two growth profiles are shown (133, 134), where the growth profile 134 corresponds to a photoautotrophic organism grown a greater light intensity than growth profile 133. At a given time, growth profile 134 shows a higher concentration of photoautotrophic organism than 133, as seen by data points 137 and 136.

### EXAMPLE 14

### Example of a Manufacturing Plant for Producing Electricity and Biomass

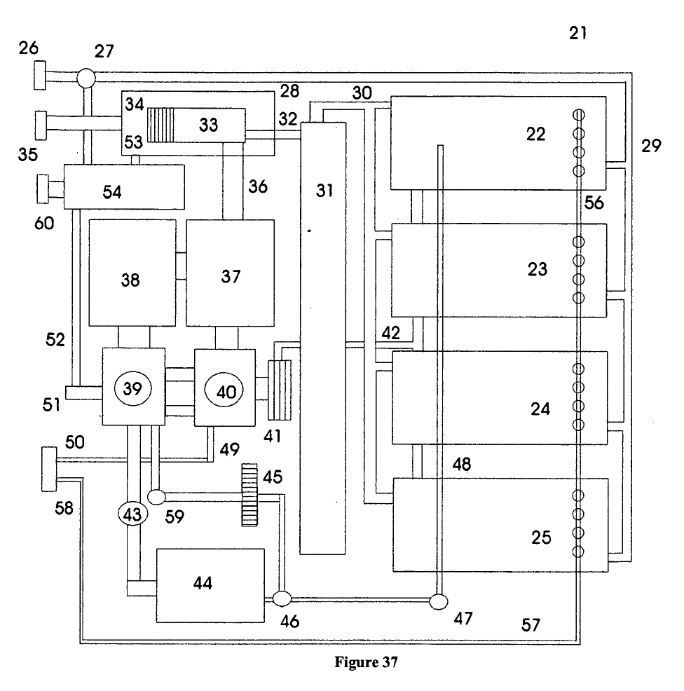

FIG. 37 shows an example of a system for producing electricity using four bioreactors in parallel (22-25) with a combustion device. The system comprises four bioreactors (22-25), a biomass extractor (33), a combustion device (39), and a steam turbine (40). Components of the system are listed as follows: a water intake (26), a water and nutrient control pump and valve (27), pipes for water distribution to the four bioreactors (29), pipes for delivering photoautotrophic organisms grown in the bioreactors and suspended in a growth media (30), a slurry tank (31) for holding photoautotrophic organisms grown in the bioreactors, a pipe and valve (32) for controlling transfer of the slurry to a hydraulic ram press, a hydraulic ram press (33) for extracting liquids from the photoautotrophic organisms, such as straight vegetable oil, a separation tank (34) for separating immiscible liquids, such as oil and water, a pump (35) for removing straight vegetable oil or other biomass products from the separation tank, a transfer means (36) for removing press cake from the hydraulic ram, a dryer (37) for evaporating entrained liquid from the press cake, a fuel hopper (38) for storing dried press cake, a combustion device (39) for burning dried press cake and other biomass, a steam turbine (40) for producing electricity from heat generated by the combustion device, a condenser (41) that can be used to collect steam generated during the electricity production process, a heat-exchanger for directing heat that is unused by the steam turbine to other processes requiring heat, such as bioreactors, a compressor (43) for collecting carbon dioxide and other flue gases produced during combustion of the press cake and other biomass, a storage tank (44) for storing carbon dioxide and other flue gases produced by combustion of the press cake and other biomass, an air intake (59) for delivering air to the combustion device and the bioreactors, a air preparation device (45) for preparing gas for delivery to the bioreactors, such as an air filter and air intake assembly, a control valve (46) for controlling ratios of carbon dioxide and air to be fed to the bioreactors, a pressure relief valve (47) for controlling gas pressure in the bioreactors, sparging manifolds (48) in each of the four bioreactors for delivering gas, an electrical conductor (49) for transferring electricity produced by the steam turbine, a transformer (50) for conditioning the electricity, a means to remove ash (51) from the combustion device, a pre-mixing tank (54) for holding water and nutrients, a means to transfer the ash (52) to the pre-mixing tank, a port (53) on the separation tank (34) for removal of water and delivery to the pre-mixing tank, an array of light sources (56) for delivering light to the bioreactors, electrical connections (57) for powering the array of light sources, and hardware (58) for delivering electricity to the electrical connections.

In some embodiments of the invention, the steam turbine can be rated at 400 kW continuous output and the combustion device can burn one ton/hour. Heat generated or in excess can be delivered throughout the bioreactor for use by other processes. For example, heat from condenser can be used by evaporator. Biomass products can be used for any purpose described herein.

### EXAMPLE 15

### Growth and Harvest of a Photoautotrophic Organism and Extraction of Biomass Products

FIG. 38 to FIG. 46 show a sequence for growing a photoautotrophic organism in a bioreactor, harvesting the photoautotrophic organism, and extracting biomass products from the photoautotrophic organism.

FIG. 38 shows a bioreactor vessel (1) comprising light conducting channels (2), a gas sparger (3), a gas intake port (4), a water and nutrient intake port (5), a harvest port (6), a holding tank (7), a biomass extractor (8), a separation tank (9), a recycle junction (13), a water intake port (14), and a movable unit (16) of a biomass collector. The light conducting channel can be used for the distribution and transmission of light into the bioreactor from one or more light sources. The biomass extractor can be a hydraulic ram that compresses biomass for separation of liquid products and solid biomass (12). The liquid products that can comprise two immiscible liquids can be further separated in the separation tank. The two immiscible liquids can be an aqueous liquid and a non-aqueous liquid. The non-aqueous liquid can comprise oil or straight vegetable oil. The aqueous liquid can be a lower fraction in the separation tank (11) and the non-aqueous liquid can be a higher fraction in the separation tank (10). The position of the aqueous liquid and non-aqueous liquid can depend on the relative densities of the two liquids.

FIG. 39 shows the movable unit (16) of the bioreactor in a lower position relative to that shown in FIG. 38.

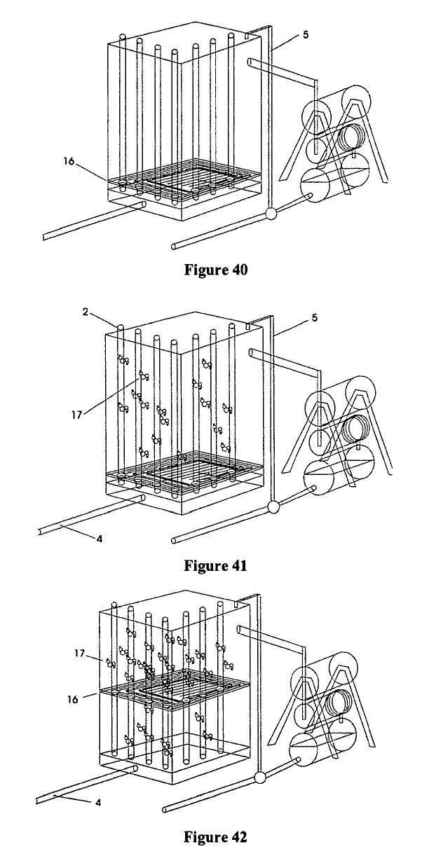

FIG. 40 shows the movable unit (16) of the bioreactor in a lowest position. During the lowering of the movable unit from the position shown in FIG. 38 to FIG. 39 to FIG. 40, water and nutrients can be supplied through the water and nutrient intake port (5) to form a growth media.

FIG. 41 shows that a photoautotrophic organism (17) can be inoculated in the growth media. Water and nutrient supply can be shut down and gases, such as carbon dioxide can be supplied through the gas intake port (4). An array of lights can be energized to supply light to the light conducting channels (2).

After a desired amount of growth of the photoautotrophic organism, the photoautotrophic organism can be harvested. FIG. 42 shows a snapshot of the harvesting process. Gas supplied to the bioreactor can be switched to an air only supply and the movable unit (16) can be raised such that the photoautotrophic organism is concentrated on the upper side of the movable unit. Movement of the movable unit can also clean the light conducting channels using cleaning elements attached to the movable unit.

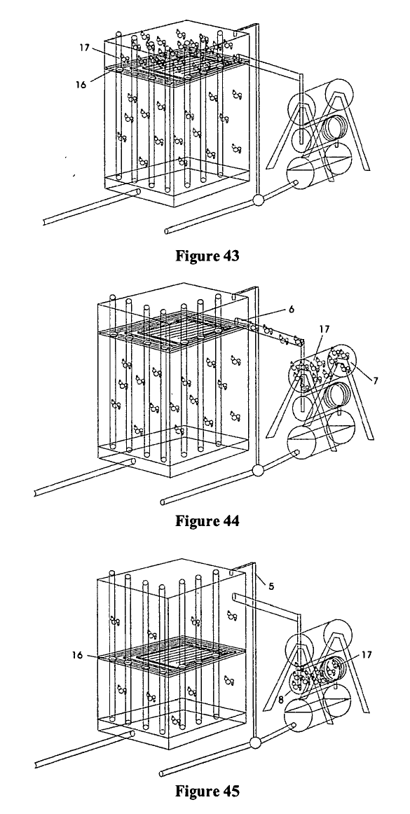

Once the movable unit reaches a highest position, as shown in FIG. 43, the photoautotrophic organism is concentrated on the top side of the movable unit forming a solution of concentrated photoautotrophic organism. A portion of the photoautotrophic organism can remain in the bioreactor for seeding of a following round of photoautotrophic organism growth.

The concentrated photoautotrophic organism, which is suspended in growth media, can be harvested through the harvest port (6), as shown in FIG. 44. The concentrated photoautotrophic organism can be transferred to the holding tank (7) for settling of the photoautotrophic organism. The holding tank can be used for gravity-based separation of the photoautotrophic organism from the growth media to form a solution of further concentrated photoautotrophic organism. Recovered growth media can be recycled back to the bioreactor.

As shown in FIG. 45, the further concentrated solution photoautotrophic organism can be transferred to the biomass extractor (17). By any extraction means known to those skilled in the arts, the photoautotrophic organism can be separated into liquid and solid biomass products. At the same time, the movable unit (16) can be lowered.

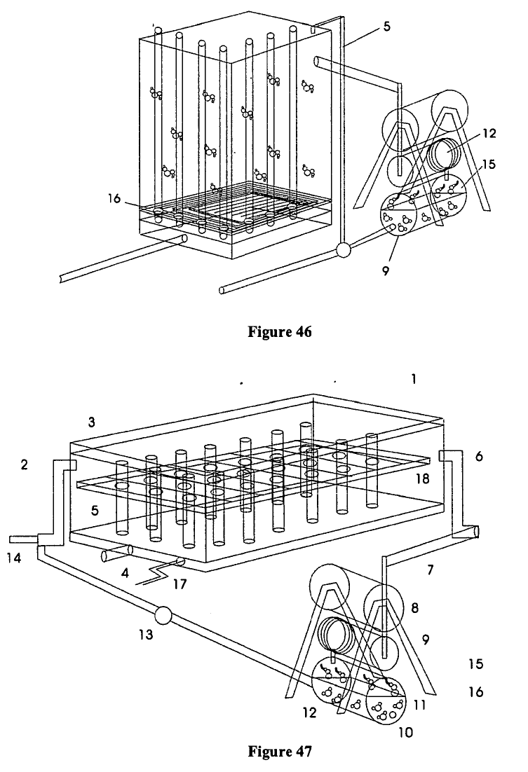

As shown in FIG. 46, the liquid biomass products can be transferred to a separation tank for separation of aqueous and non-aqueous liquid biomass products. The aqueous biomass products can be diluted in growth media not separated from the photoautotrophic organism in previous steps. In some embodiments of the invention, the growth media that was collected after extraction of biomass products is returned to the bioreactor.

### EXAMPLE 16

### Example of an Assembly of Light Conducting Channels and Gas Sparging Devices

FIG. 47 shows an example of a system comprising a bioreactor (1) with a biomass collector (18) and a biomass processing system including a holding tank (8), a biomass extractor (9), and a separation tank (1). Additional components of the system include a water and nutrient intake (2), an LED array and light conducting channels (3), a CO₂ and air sparging intake (4), an interior volume (5), an export valve (6), an export pipe, a pump (13), an import pipe (14), and a power supply (15).

The bioreactor can have dimensions that are approximately twenty by twenty by forty feet. The total volume of the bioreactor can be approximately 12,800 cubic feet. The water and nutrient intake can be used for supplying a growth media to the bioreactor. The LED array and light conducting channels can be used for delivery of light and stimulation of photosynthesis in photoautotrophic organisms growing in the bioreactor. In approximately 20 hours, the photoautotrophic organisms can have a concentration that at least doubles. The photoautotrophic organisms can be harvesting using the biomass collector, as described herein. The biomass collector can concentrate at least 50% of the photoautotrophic organisms on one side of the biomass collector in a volume corresponding to at most 20% of the bioreactor, forming a solution of concentrated photoautotrophic organisms. The export valve can allow for delivery of the solution of concentrated photoautotrophic organisms to the holding tank, and the biomass extractor can be used to extract liquid biomass products from the photoautotrophic organisms, such as oils, press cake, and other biomass products. The liquid biomass products and growth media can be delivered from the biomass extractor to the separation tank for separation of aqueous biomass products and growth media (10) and non-aqueous biomass products (11). The pump (13) can be used to return the growth media separated by the separation tank to the bioreactor. The press cake can be combusted to produce electrical energy using a generator.

The energy requirements can be as follows: powering LED array and components for nutrient/gas supply during growth—10 kW×20 h=200 kWh, powering other components during growth—40 kW×20 h=800 kWh, powering air only supply during harvest—10 kW×4 h=40 kWh, powering biomass extraction—40 kW×2 h=80 kWh, extraction of oil from separation tank—5 kW×2 h=10 kWh, pumping 20% of the volume of the bioreactor to replace lost growth media—5 kW×4 h=20 kWh. The total energy cost can be 1,150 kWh per cycle. At 10 cents per kWh, this corresponds to approximately $115 per cycle.

The estimated production of biomass products is 686 gallons of oil and 12,500 pounds of press cake.

### EXAMPLE 17

### Bioreactor with Multiple Optical Elements

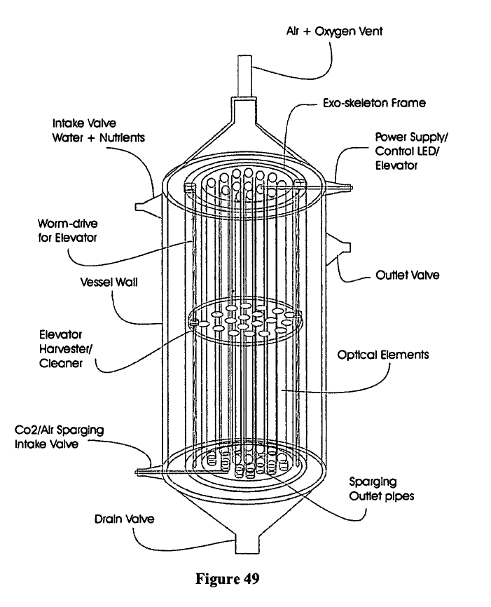

FIG. 49 shows an example of a bioreactor with multiple optical elements and an elevator harvester/cleaner. The optical elements are light conducting channels that allow for distribution of light emitted from one or more light sources. The one or more light sources can be positioned at an end of the light conducting channels. The array of light sources can be placed at a top end or bottom end of the light conducting channels. The elevator harvester/cleaner can be a biomass collector described herein that can be used to concentrate a photoautotrophic organism grown in the bioreactor and to clean the optical elements. The elevator harvester/cleaner can be mechanically connected to a worm drive such that the worm drive allows for movement of the elevator harvester/cleaner. As shown in FIG. 49, additional components of the bioreactor can include an air and oxygen vent for releasing excess gas from the bioreactor, an exo-skeleton frame for supporting the optical elements, an outlet valve for harvesting materials from the bioreactor, sparging outlet pipes for supplying the bioreactor with gases, such as carbon dioxide and air, a drain valve for removing materials from the bioreactor, a carbon dioxide and air sparging intake valve for supplying gas to the bioreactor, a vessel wall that is substantially water tight, and an intake valve for supplying water and nutrients.

### EXAMPLE 18

### Bioreactors with Containers as Light Conducting Channels

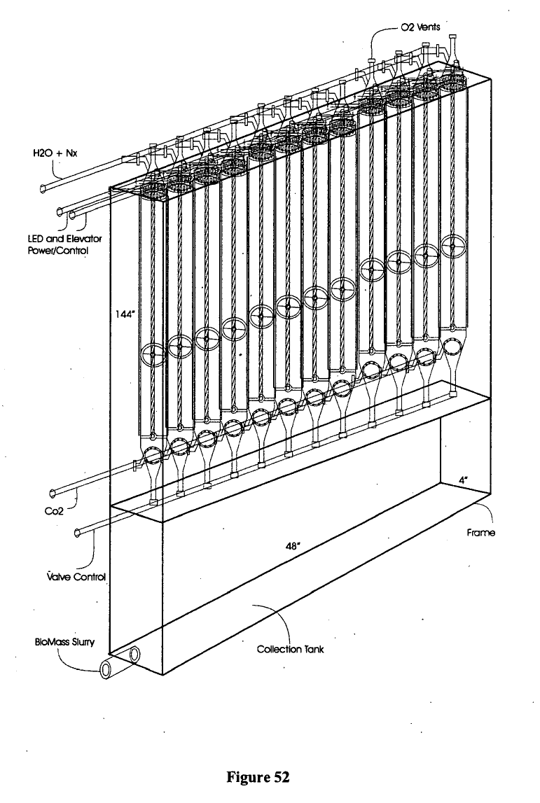

FIG. 50, FIG. 51, and FIG. 52 show additional embodiments of bioreactors with containers that are light conducting channels. The containers are manufactured of materials that are optically transparent, such as glass, acrylic, or any other polymer known to those skilled in the arts. The containers are coated or surrounded by a reflector for retaining light within the bioreactor. A reflector can also be placed on a bottom side of the bioreactor for retaining light within the bioreactor. The bioreactor can comprise a LED array that is positioned at a top edge of the container, such that light emitted by the LED array is directed into walls of the container. The light that is directed into the walls of the container can be transmitted down the walls of the bioreactor until the light is distributed into an interior space of the bioreactor, which is determined by an incident angle of light from the LED array into the walls of the bioreactor. Light emitted by the LED array can be incident on the reflector and then be directed into the interior space of the bioreactor.

As shown in FIG. 50, the bioreactor can comprise an elevator cleaner. The elevator cleaner can comprise one or more cleaning elements described herein. The elevator cleaner can be mechanically connected to a worm drive shaft for moving the elevator cleaner along a height of the bioreactor. In some embodiments of the invention, the worm drive shaft is optically transparent and can be a light conducting channel. An LED array can also be positioned at a top edge of the worm drive shaft such that light emitted by the LED array is directed into the worm drive shaft.

Additional components of the bioreactor are shown in FIG. 50. These additional components include a bracket for supporting the worm drive shaft, a water and nutrient input port for supplying water and nutrients, a gas vent for releasing gas from the bioreactor, electrical connections for powering and controlling one or more LED arrays, electrical connections for powering and controlling the worm drive shaft, a drain at the bottom side of the bioreactor for removing contents from the bioreactor, and a gas input port for supplying the bioreactor with gases, such as carbon dioxide and air.

FIG. 51 shows an illustration of an array of bioreactors with nine bioreactors. The bioreactors can be similar to the bioreactor depicted in FIG. 50. Each bioreactor has a cylindrical container that is approximately 4 inches in diameter and 12 feet in height. The total volume of the cylindrical container is approximately 1 cubic foot. The amount of liquid that can be contained is approximately 62 pounds. One bioreactor has a 12 square foot surface area for distribution of light and a 12 square inch footprint. One square foot can have nine such bioreactors. The bioreactors can share electrical connections and have fluidly connected inputs and outputs. The total volume can be approximately 9 cubic feet with a total mass of about 560 pounds. The output of the array of bioreactors can be in the range of 0.3 to 30 grams of biomass per liter or 0.019 to 1.9 pounds of biomass per cubic feet. The estimated production of biomass is in the range of 3 to 30 grams per liter or 0.19 to 1.9 pounds per cubic feet of biomass. The expected biomass productivity is at least one pound of biomass per cubic feet per day. Accordingly, the expected productivity of the array of bioreactors is 9 pounds per day. For a hypothetical one hundred square foot pace, the productivity of biomass can be approximately 900 pounds of biomass per day. Further scaling this to one acre of space, the productivity can be approximately 392,000 pounds of biomass per day.

FIG. 52 shows an array of bioreactors with a collection tank harvesting biomass from the bioreactors. The array of bioreactors can comprise 12 bioreactors with a total working volume of 12 cubic feet. The bioreactors have a total amount of surface area for distribution of light that is approximately 144 square feet. The aerial footprint of the array of bioreactors can be approximately 1.3 square feet.

The bioreactors can have inputs and outputs that are fluidly connected for efficient delivery and removal of materials to and from the bioreactors. The fluidly connected inputs can be inputs for water, nutrients and carbon dioxide. The fluidly connected outputs can be outputs for oxygen and growth media containing damaged or dead photoautotrophic organisms grown in the bioreactor. The output for the growth media can be placed at a bottom side of the bioreactor such that the growth media containing damaged or dead photoautotrophic organisms is transferred from the bioreactors to a collection tank. The collection tank can be used for gravity separation of the damaged or dead photoautotrophic organisms from the growth media. The damaged or dead photoautotrophic organisms can settle at the bottom of the collection tank and exit the collection tank through a biomass slurry port.

1-79. (canceled)

80. A bioreactor comprising:
a) a container for culturing a photoautotrophic organism, said photoautotrophic organism having at least one light absorption pigment, the at least one light absorption pigment having one or more peak absorption wavelengths; and
b) a light source configured to emit one or more wavelengths of light reaching said container, wherein the one or more wavelengths of light are adjustable based on a growth profile of said photoautotrophic organism; and
c) a light conducting channel operably linked to said light source, wherein said light conducting channel has a surface area that distributes light from at least about 50% of exterior surface area of said channel.

81. The bioreactor according to claim 80, wherein said wavelengths of light is to be adjusted for intensity, wavelength, duration, and frequency, and wherein the photoautotrophic organism is selected from the group consisting of algae, bacteria, euglena, diatom, phytoplankton, botryococcus braunii, chlorella, and dunaliella.

82. The bioreactor according to claim 80, further comprising a gas sparger with holes less than approximately 0.01, 0.05 0.1, 0.25, 0.5, or 1 cm in diameter configured to deliver a gas to the bioreactor.

83. The bioreactor according to claim 80, wherein the light source is selected from the group consisting of a light emitting diode, a laser, an incandescent light bulb, and a gas discharge bulb.

84. The bioreactor according to claim 80, wherein the light conducting channel is placed in the interior of said container, and optionally wherein the light conducting channel comprises a polymer and a reflective element.

85. The bioreactor according to claim 80, further comprising an energy converter for production of electrical energy from a renewable energy source, wherein said energy converter is operably linked to said light source.

86. The bioreactor according to claim 80, further comprising a light-receiving element configured to receive solar light for culturing the photoautotrophic organism.

87. The bioreactor according to claim 80, wherein the light source comprises an array of light emitters.

88. The bioreactor according to claim 80, further comprising a power plant operably linked to said bioreactor, wherein said power plant converts said biomass to electricity and carbon dioxide, wherein said carbon dioxide is supplied to said bioreactor for production of said biomass.

89. A method of producing biomass using a bioreactor according to claim 80 comprising:
culturing a photoautotrophic organism in a medium contained in the bioreactor, wherein a light source is configured to yield a biomass production efficiency at no less than about 50 milligrams of said biomass per kJ of energy that is supplied to the light source.

90. The method of producing biomass according to claim 89 further comprising:
culturing the photoautotrophic organism in the medium contained in the bioreactor under conditions such that more than about 30, 50, 75, or 200 grams of biomass per liter of medium are produced.

91. A method of producing biomass using a bioreactor according to claim 80 further comprising:
a) introducing a photoautotrophic organism to the bioreactor, wherein the bioreactor comprises a container operably linked to a light source that is configured to emit at least one or more wavelengths of light reaching said container;
b) determining a growth profile or biomass production rate of said phototrophic organism; and
c) adjusting the at least one or more wavelengths of light based on results of step b).

92. The method of producing biomass according to claim 89 further comprising:
a) producing electrical energy from a renewable energy source; and
b) utilizing said electrical energy to power said light source.

93. The method of producing biomass according to claim 92 further comprising maintaining growth of said photoautotrophic organism using an artificial light source and solar light from a light-receiving element.

94. The method of producing biomass according to claim 92 further comprising:
a) supplying electricity and carbon dioxide to a bioreactor for production of said biomass; and
b) using a power plant for producing said electricity and carbon dioxide from said biomass.

95. The method of producing biomass according to claim 92, wherein the light source intensity, wavelength, duration, and/or frequency is adjusted based on the growth profile of the photoautotrophic organism or biomass production rate.

96. The method of producing biomass according to claim 92, further comprising transmitting the one or more wavelengths of light emitted by the light source through the light conducting channels and into the bioreactor.
